(19) United States
(12) Patent Application Publication
(10) Pub. No.: US 2009/0137002 A1
(43) Pub. Date: May 28, 2009

(54) ANTI EPHB4 ANTIBODIES AND ANTIBODY FRAGMENTS

(76) Inventors: Heike PETRUL, Berlin (DE); Andreas Menrad, Ely Cambs (GB); Jorg Willuda, Berlin (DE); Dieter Zopf, Berlin (DE); Jens Glienke, Berlin (DE); Josef Prassler, Germering (DE); Andreas Popp, Kaufering (DE); Stefan Steidl, Munchen (DE); Catrin Pracht, Neuried (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/182,235

(22) Filed: Jul. 30, 2008

Related U.S. Application Data
(60) Provisional application No. 60/953,771, filed on Aug. 3, 2007.

(30) Foreign Application Priority Data
Jul. 31, 2007 (EP) 07075657.2

(51) Int. Cl. C12P 21/02; A61K 39/395; C07H 21/02; A61P 35/00; C12N 15/63; C12N 5/10

(52) U.S. Cl. 435/69.6; 530/388.22; 424/158.1; 536/23.53; 435/320.1; 435/334

(57) ABSTRACT

The invention concerns antibodies and antibody fragments, specifically recognising the EphB4 receptor comprising heavy chains and light chain, comprising light and heavy CDR's, and their use as medicament for the treatment of pathological angiogenesis, in particular cancer therapy.

This application claims the benefit of the filing date of U.S. Provisional Application Ser. No. 60/953,771 filed Aug. 3, 2007.

The invention relates generally to antibodies and antibody fragments specifically recognizing the receptor EphB4 and to methods of inhibiting angiogenesis and/or tumor growth in a mammal using said antibodies and antibodies and antibody fragments.

The process of angiogenesis, by which new blood vessels develop from existing ones by branching and sprouting, is crucial for vascular remodeling in embryogenesis and normal tissue homeostasis (e.g., female reproductive cycle). Angiogenesis also plays an important role in the pathogenesis of diseases such as cancer and retinopathy. The pro-angiogenic environment of these diseases results in new blood vessels which allow the development and the maintenance of the pathological state.

The Eph family of receptor tyrosine kinases and their ligands, the ephrins, are critical regulators of vascular remodeling in the embryo, postnatal vascular remodeling, and tumor neovascularization. With its currently known 15 members, the Eph family represents the largest family of receptor tyrosine kinases and is divided into the A and the B classes based on sequence homology and the structural differences of the ligands. The membrane-attached ligands differ in the transduction of the signal into the cell: The A ephrins are linked to the cell via a glycosylphosphatidylinositol (GPI) anchor, whereas the B ephrins have a transmembrane region and a short intracellular domain.

The Eph receptors and ephrins mediate bi-directional signaling: forward signaling through the Eph receptor and reverse signaling via the ephrin ligand. In general, nine different EphA receptors (EphA1-A9) promiscuously interact with six A-ephrins (EphrinA1-A6), whereas the receptors of the B-subclass (EphB1-B6) interact with three different B-ephrins (ephrin B1-B3). The EphB4 receptor (“EphB4”), however, only binds the ligand EphrinB2, making this one-receptor-one-ligand interaction an interesting target for therapeutic approaches.

EphB4 and its ligand EphrinB2 are expressed in the developing central nervous system, where they function in contact-mediated axon guidance, axon fasciculation, and guided cell migration. EphrinB2 and EphB4 mediate spatial organization signalling during angiogenesis and vessel assembly. In other contexts, however, Eph/Ephrin signalling appears to enhance cellular adhesion, such as in a human breast cancer model in mice (Noren et al., Proc. Natl. Acad. Sci. USA 101, 5583-88, 2004). In this model, EphB4 on the tumour cells attracts EphrinB2-bearing endothelial cells. As a result, the tumour is better vascularized and therefore grows faster than the control. Interestingly, this model uses a kinase-deleted mutant of EphB4 which shows that the (indirect) growth advantage of the tumour cells is not dependent on the EphB4 kinase domain, but rather on the EphB4 ectodomain which interacts with EphrinB2. In light of these results, blocking of the interaction between EphB4 and the ligand EphrinB2 should lead to tumor growth inhibition. There is, therefore, a need in the art for molecules which can perform this function with high specificity and affinity.

The problem of the invention is to find an antibody or antibody fragment specifically recognising the EphB4 receptor with a high effectiveness. Further the antibodies or antibody fragments can be used as a medicament especially for treatment of cancer, for example breast cancer.

Known IgG and IgM molecules (e.g. WO 2004/080425 A2) against EphB4 extracellular domain, cited in the literature of patents and science, are not sufficiently described and are not available to the scientific community and are not described sufficiently for a teaching to create pre published state of the art. On page 63, line 5 to 11 the manufacture of anti EphB4 monoclonal antibodies is described which is an insufficient disclosure. Further on page 99 the use of anti EphB4 monoclonal antibodies are mentioned. In both text passages the amino acid sequences and/or the deposition of the monoclonal antibodies is lacking.

The problem is solved by an antibody or antibody fragment specifically recognizing the EphB4 receptor comprising one or more heavy and light chain sequences selected from the sequences of Seq. ID NO. 1 to Seq. ID NO. 118, or modifications thereof, whereby the antibodies or antibody fragments comprise a complementarity determining region (CDR) of a heavy chain (CDR-H) and a light chain (CDR-L). The heavy chain (CDR-H) is, for example, selected from the group of Seq. ID NO. 1 to 3, Seq. ID NO. 7 to 9 and Seq. ID NO. 13 to 15; and the light chain (CDR-L) is, for example selected from the group of Seq. ID NO. 4 to 6, Seq. ID NO. 10 to 12, and Seq. ID NO. 16 to 18.

The antibodies or antibody fragments may comprise the heavy chain (CDR-H), with CDR-H1 selected from the group of Seq. ID NO. 1, Seq. ID NO. 7 and Seq. ID NO. 13, or as CDR-H2 from the group of Seq. ID NO. 2, Seq. ID NO. 8 and Seq. ID NO. 14, or as CDR-H3 from the group of Seq. ID NO. 3, Seq. ID NO. 9 and Seq. ID NO. 15 and the light chain (CDR-L) as CDR-L1 from the group of Seq. ID NO. 4, Seq. ID NO. 10 and Seq. ID NO. 16, or as CDR-L2 from the group of Seq. ID NO. 5, Seq. ID NO. 11 and Seq. ID NO. 17, or as CDR-L3 from the group of Seq. ID NO. 6, Seq. ID NO. 12 and Seq. ID NO. 18.

An object of the instant invention are further antibodies or antibody fragments, which comprise a combination of the above mentioned heavy chains (CDR-H) together with the above mentioned light chains (CDR-L).

The inventive antibodies or antibody fragments may further comprise the variable domain (V) of the complementarity determining region (CDR) of a heavy chain (CDR-H, VH) and a light chain (CDR-L, VL).

The heavy chain (CDR-H) may be selected from the group of Seq. ID NO. 19 and the light chain (CDR-L) may be selected from the group of Seq. ID NO. 20, or the heavy chain (CDR-H) may be selected from the group of Seq. ID NO. 21 and the light chain (CDR-L) max be selected from the group of Seq. ID NO. 22, or the heavy chain (CDR-H) may be selected from the group of Seq. ID NO. 23 and the light chain (CDR-L) may be selected from the group of Seq. ID NO. 24.

However, these combination examples are just a small number of possible combinations, and it is thus noted that all inventive sequences, such as for example the CDR's, can be combined with each and every inventive sequence, in all possible ways. These combinations are also comprised by the instant invention.

The instant invention also concerns combinations of all sequences. For example, the instant invention concerns those antibodies and antibody fragments, specifically recognising the EphB4 receptor comprising a complementary determining region (CDR) of a heavy chain and a light chain wherein the CDR of the heavy chain (CDR-H) comprises the sequence of the amino acids of Seq Id No 1 (3640 CDR-H1) and Seq Id No 2 (3640 CDR-H2) and Seq Id No 3 (3640 CDR-H3) or modifications thereof, or the Seq Id No 7 (3639 CDR-H1) and Seq Id No 8 (3639 CDR-H2) and Seq Id No 9 (3639 CDR-H3) or modifications thereof, or the Seq Id No 13 (3641 CDR-H1) and Seq Id No 14 (3641 CDR-H2) and Seq Id No 15 (3641 CDR-H3) or modifications thereof, and/or wherein the CDR of the light chain (CDR-L) comprises the sequence of the amino acids of the Seq Id No 4 (3640 CDR-L1) and Seq Id No 5 (3640 CDR-L2) and Seq Id No 6 (3640 CDR-L3) or modifications thereof, or the Seq Id No 10 (3639 CDR-L1) and Seq Id No 11 (3639 CDR-L2) and Seq Id No 12 (3639 CDR-L3) or modifications thereof, or the Seq Id No 16 (3641 CDR-L1) and Seq Id No 17 (3641 CDR-L2) and Seq Id No 18 (3641 CDR-L3) or modifications thereof; and those antibodies or antibody fragments, specifically recognising the EphB4 receptor comprising a CDR of a heavy chain comprising CDR-H1, CDR-H2 and CDR-H3 and a light chain comprising CDR-L1, CDR-L2 and CDR-L3 wherein one of the heavy chains comprising the CDR-H1 selected from group Seq Id No 1, Seq Id No 7, Seq Id No 13 or a modification thereof and the CDR-H2 selected from the group Seq Id No 2, Seq Id No 8, Seq Id No 14 and the CDR-H3 selected from the group Seq Id No 3, Seq Id No 9, Seq Id No 15 or modifications thereof, and/or wherein one of the light chains comprising the CDR-L1 selected from the group Seq Id No 4, Seq Id No 10, Seq Id No 16 or modifications thereof and the CDR-L2 selected from the group Seq Id No 5, Seq Id No 11, Seq Id No 17 or modifications thereof and the CDR-L3 selected from the group Seq Id No 6, Seq Id No 12, Seq Id No 18 and modifications thereof; and antibodies and antibody fragments, specifically recognising the EphB4 receptor comprising Seq Id No 19 (variable domain 3640 CDR-H) and Seq Id No 20 (variable domain 3640 CDR-L), or Seq Id No 21 (variable domain 3639 CDR-H) and Seq Id No 22 (variable domain 3639 CDR-L), or Seq Id No 23 (variable domain 3641 CDR-H) and Seq Id No 24 (variable domain 3641 CDR-L) or modifications thereof; and antibodies or antibody fragments specifically recognising the EphB4 receptor comprising a heavy chain selected from the group Seq Id No 19 (3640 variable domain of CDR-H) and Seq Id No 21 (3639 variable domain of CDR-H) and Seq Id No 23 (3641 variable domain of CDR-H) and modifications thereof, and a light chain selected from the group Seq Id No 20 (3640 variable domain of CDR-H) and Seq Id No 22 (3639 variable domain of CDR-H) and Seq Id No 24 (3641 variable domain of CDR-H) and modifications thereof; and antibodies or antibody fragments specifically recognising the EphB4 receptor comprising Seq Id No 25 (3640 CDR-H/C), Seq Id No 26 (3640 CDR-L/C) or Seq Id No 27 (3639 CDR-H/C), Seq Id No 28 (3639 CDR-L/C) or Seq Id No 29 (3641 CDR-H/C), Seq Id No 30 (3641 CDR-L/C) or modifications thereof, and antibodies and antibody fragments specifically recognising the EphB4 receptor comprising heavy chain (CDR-H/C) selected from the group Seq Id No 25 (3640), Seq Id No 27 (3639), Seq Id No 29 (3641) and a light chain (CDR-L/C) selected from the group Seq Id No 26 (3640), Seq Id No 28 (3639), Seq Id No 30 (3641) and modifications thereof.

Antibodies against the EphB4 receptor are generally described. The disadvantage of the known antibodies is characterised in the function of the antibodies, but, however, no sequence has ever been described. On the other hand, no antibody has been deposited at any cell culture collection. Thus, as a matter of fact, those antibodies are not reproducible, and thus there is a demand for reproducible antibodies and antibody fragments.

Those commercially available antibodies against the EphB4 receptor are only characterized by the binding function, and neither an in vivo function nor any sequence is disclosed.

The production of a functional and usable antibody or antibody fragment is only possible if the single sequences are known, so that the light and heavy chains can be combined to form said functional and usable antibodies or antibody fragments.

Even thought the skilled person in that field could easily find out the sequence listing by using standard methods (e.g. Edman degradation), which on the other hand would be an undue burden for the skilled person, there is still a strong demand for further antibodies and antibody fragments having a defined sequence and a defined biological function. If both features, the defined sequence and the defined biological function are known to the artisan/those skilled in the art, it will be possible to create functional antibodies and antibodies and antibody fragments (monomers and dimmers) from the defined and well selected light or heavy chains

Surprisingly, the antibodies and antibody fragments of the instant invention have in fact the advantage of being characterised by sequence and functional features. Surprisingly in addition, it has further been found that the monomers (for example Fab′) and the dimers (for example F(ab′)₂) are blocking the interaction of EphrinB2 in cells, especially in endothelial cells. It has further been found that only the dimers induce the intracellular kinase activity of EphB4.

A further embodiment of the invention is an antibody or antibody fragment which exhibits a substantially equivalent antigen binding to the EphB4 receptor,

or
an antibody or antibody fragment which binds to the same epitope of the EphB4 receptor.

Substantially equivalent antigen binding or the same epitope binding can be determined by competitive inhibition kinetic of one of the instant antibodies or antibody fragments, especially the inventive antibodies and antibody fragments of FIG. 6.

When using the instant monomeric antibodies and antibody fragments or the dimeric antibodies and antibody fragments, the phosphorylation inhibition (see examples 3 and 4) are indicators for the substantially equivalent antigen binding or the binding to the same epitope.

Other techniques which show a competitive binding of an anti-EphB4 antibody and an EphrinB2 ligand include competitive ELISA or Biacore™.

The heavy and the light chain can be combined by dimerisation. Dimerisation tools are well described in the literature. Examples of these dimerisation tools are, for example, peptide linkers, disulfide bridges, double helices. Typical realisations are Fab's (named “monomers” in the instant invention, since they comprise one heavy and one light chain (each consisting of a variable and a constant fragment and forming one antigen binding site), and F(ab′)₂ (named “dimers” in the instant invention, since they comprise two realisations of the variable fragment and the constant fragment of the heavy and the light chain, both connected via disulfide bridges).

If the disulfide bridges concern a peptide antibody-linker, the heavy chain might be N-terminal and the light chain might be C-terminal or the heavy chain might be C-terminal and the light chain might be N-terminal.

The sequence of the antibody linker is described in EP 0 573 551; EP 0 318 554 and EP 0 623 679.

The antibodies and antibody fragments according to the instant invention can be used in pathological angiogenesis in particular cancer therapy, but also for the treatment of diabetic retinopathy, etc.

The antibodies and antibody fragments according to the instant invention can be combined with any active cancer therapeutical compound.

Further, the invention comprises DNA sequences, encoding antibodies and antibody fragments, specifically recognising the EphB4 receptor, wherein the amino acid sequence comprises combinations and sequences as mentioned above.

Additionally, the invention provides fully human antibodies and antibody fragments which bind to the extracellular domain of EphB4.

Preferred antibodies and antibody fragments have been identified by screening a synthetic phage display antibody library (HuCAL®; Knappik et al., J. Mol. Biol. 296, 57-86, 2000). The antibodies and antibody fragments of the instant invention can be used to inhibit the binding of EphrinB2 to EphB4, thereby inhibiting activities mediated by EphB4 and by EphrinB2.

The antibodies and antibody fragments are useful for a variety of purposes, particularly for treating diseases characterized by abnormal angiogenesis, e.g. an increased angiogenesis, such as cancer. Preferably the antibodies and antibody fragments of the invention are capable of modulating, e.g. inhibiting or stimulating EphB4 phosphorylation in vivo. Phosphorylation of EphB4 can be detected using any suitable means known in the art. See, e.g., Noren et al., 2005, or Palmer et al., Mol. Cell. 2002 April; 9(4), 725-37. WO 04/080425 describes a cell-based EphB4 tyrosine kinase assay.

Further, the present invention refers to antibodies and antibody fragments which bind to the same epitope on the EphB4 receptor as one of the antibodies and antibody fragments MOR 03639, MOR 03640, MOR 03641, or all other instant antibodies. The epitopes to which the antibodies and antibody fragments bind may be determined by cross-blocking and/or epitope mapping according to standard methods.

The antibodies and antibody fragments specifically recognize the EphB4 receptor, particularly the human EphB4 receptor. An ELISA assay can be carried out to identify antibodies and antibody fragments which cross-react with human and, e.g., mouse EphB4 with high affinity and specificity. Functional cross-reactivity can be confirmed using, for example, a phosphorylation assay. Therapeutic utility of the antibodies and antibody fragments can then be tested in vivo in an animal model.

Antibodies and antibody fragments of the instant invention bind to an epitope of EphB4 and preferably interfere with the binding to EphrinB2, which can lead to an inhibition of the EphB4-mediated signalling.

Monomeric anti-EphB4 antibodies and antibody fragments of the instant invention can block the EphrinB2-induced phosphorylation of EphB4, which itself will block the kinase activity of the receptor (see FIG. 2). Further, the antibodies and antibody fragments in the dimeric format can stimulate the phosphorylation of EphB4 (see FIGS. 3 and 18).

The term “combination” as used in the instant application means a pharmaceutical composition comprising at least two pharmaceutically effective compounds. These compounds can be administered simultaneously, frequently, or sequentially. The compounds can be administered via the same route or in a different manner. Thus, the compounds can be applied orally and/n or parenterally.

The term “composition” as used in the instant application means at least one pharmaceutically effective compound and further pharmaceutically acceptable diluents and/or carriers.

The term “fusion protein” as used in the instant application means a protein comprising different amino acid sequences which are defined by the origin and/or by special functions.

EphB4 epitopes specifically recognised by antibodies are not described in the state of the art.

Antibody linker are described in EP 0 573 551; EP 0 623679 and EP 0 318554, which documents are introduced by reference.

Fusion protein linker are described in EP 0 573 551; EP 0 623679 and EP 0 318554, which documents are introduced by reference.

Active compounds which can be used in cancer therapy are described in WO2005/108415 and which comprise cytotoxins such as cytostatic agents, alkylating agents, antimetabolites, anti-proliferative agents, tubulin binding agents, hormones and hormone antagonists, and the like. Exemplary cytostatics that are compatible with the present invention include alkylating substances, such as mechlorethamine, triethylenephoramide, cyclophosphamide, ifosfamide, chlorambucil, busulfan, melphalan or triaziquone, also nitrosourea compounds, such as carmustine, lomustine, or semustine. Other preferred classes of cytotoxic agents include, for example, the maytansinoid family of drugs. Other preferred classes of cytotoxic agents include, for example, the anthracycline family of drugs, the vincy drugs, the mitomycins, the bleomycins, the cytotocix nucleosides, the pteridine family of drugs, diynenes, and the podophyllotoxins. Particularly useful members of those classes include, for example, adriamycin, caminomycin, daumorubicin (daunomycin) doxorubicin, aminopterin, methotrexate, methopterin, mithramycin, streptonigrin, dichloromethotrexate, mitoycin C, actinomycin-D, porfiromycin, 5-fluorouracil, floxuridine, ftorafur, 6-mercaptopurine, cytarabine, cytosine arabinoside, podophyllotoxin, or podophyllotoxin derivatives such as etoposide or etoposide phosphate, melphalan, vinblastine, vincristine, leurosidine, vindesine, leurosine and the like. Still other cytotoxins that are compatible with the teachings herein include taxol, taxane, cytochalasin B, gramicidin D, ethidium bromide, emetine, tenoposide, colchicin, dihydroxy anthracin dione, mitoxantrone, procaine, tetracaine, lidocaine, propranolol, and puromycin and analogs or homologs thereof. Hormones and hormone antagonists, such as corticosteroids, e.g. prednisone, progestins, e.g. hydroxyprogesterone or medroprogesterone, estrogens, e.g. diethylstilbestrol, antiestrogens, e.g. tamoxifen, androgens, e.g. testosterone, and aromatase inhibitors, e.g. aminogluthetimide are also compatible with the teachings herein. One skilled in the art may make chemical modifications to the desired compound in order to make reactions of the compound more convenient for purposes of preparing combination of the invention.

The term “antibodies and antibody fragments” comprise a portion of an intact antibody, preferably comprising the antigen-binding or variable region thereof.

Examples of antibodies and antibody fragments include Fab, Fab′, F(ab′)₂ and Fv fragments; diabodies; linear antibodies; single-chain antibody molecules; and multispecific antibodies formed from antibodies and antibody fragments. Examples are antibodies, native antibodies, Fab-fragments, Fab′ (here defined a monomers) and F(ab′)₂ (here defined as dimers), “Fv”, Single-chain Fv” or ‘scFv” antibodies and antibody fragments; monoclonal antibody; monoclonal antibodies” isolated from phage antibody libraries; chimeric antibodies; humanised antibodies; antibodies and antibody fragments isolated from antibody phage libraries, human antibodies.

Monomeric antibodies and antibody fragments, for example Fab fragments, are preferred. Monomeric fragments have several advantages over full IgG molecules. For example, they can better penetrate tumours and are cleared more quickly than full IgG molecules. In addition, Fab fragments can be conveniently and inexpensively produced in E. coli, considerably lowering the cost of producing the fragments. The Fab fragments described in this disclosure are easily amenable to further genetic modification. This genetic engineering includes, but is not limited to, affinity maturation of the antibodies and antibody fragments. If desired, however, EphB4 Fabs can be converted into full immunoglobulins, for example IgG1 antibodies. Method of carrying out such conversions are well known in the art. Other modifications include PEGylation to change pharmacodynamics of proteins. PEGylation is offered by commercial providers, e.g. Celares (www.celares.com)

Various techniques have been developed for the production of antibodies and antibody fragments. Traditionally, these fragments were derived via proteolytic digestion of intact antibodies (See, e.g., Morimoto et al., Journal of Biochemical und Biophysical Methods 24: 107-117 (1992) and Brennan et al., Science, 229: 81 (1985)). However, these fragments can now be produced directly by recombinant host cells. For example, the antibodies and antibody fragments can be isolated from the antibody phage libraries discussed above. Alternatively, Fab′-SH fragments can be directly recovered from E. coli and chemically coupled to form F(ab′)₂ fragments (Carter et al., Biotechnology 10: 163-167 (1992)). According to another approach, F(ab′)₂ fragments can be isolated directly from recombinant host cell culture. Other techniques for the production of antibodies and antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. The antibody fragment may also be a “linear antibody”, e.g., as described in U.S. Pat. No. 5,641,870 for example. Such linear antibodies and antibody fragments may be monospecific or bispecific.

The term “diabodies” refers to small antibodies and antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example,

EP 0 404 097; WO 93/11161; and Hollinger et al., Proc. Natl. Acad. Sci. USA, 905444-6448 (1993).

The term “antibody” herein is used in the broadest sense and specifically covers intact monoclonal antibodies, polyclonal antibodies, multispecific antibodies (e.g. bispecific antibodies) formed from at least two intact antibodies, and antibody fragments so long as they exhibit the desired biological activity.

The term “native antibodies” is usually understood as heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “variable” refers to the fact that certain Portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (See Kabat et al., Sequences of Proteins Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “Fv” is understood as the minimum antibody fragment which contains a complete antigen-recognition and antigen-binding site. This region consists of one heavy chain and one light chain variable domain in tight, non-covalent association. It is in this configuration that the three hypervariable regions of each variable domain interact to define an antigen-binding site on the surface of the V_{H}-V_{L}. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′), antibodies and antibody fragments originally were produced as pairs of Fab′₂-fragments which have hinge cysteines between them. Other chemical couplings of antibodies and antibody fragments are also known.

The variable domains comprises the 3 CDRs (hypervariable domains) and 4 framework domains (FR), flanking the CDRs. For example such a sequence will have the domains:

FR-CRD1-FR′-CDR2-FR″-CDR3-FR′′′.

The light chains of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of antibodies are called α, δ, ε, γ, and μ respectively. The subunit structures and three-dimensional configurations of different classes of immunoglobulins are well known.

Single-chainFv or scFv antibodies and antibody fragments comprise the V_{H} and V_{L} domains of antibody, wherein these domains are present in a Single Polypeptide. chain. Preferably, the Fv Polypeptide further comprises a Polypeptide linker between the V_{H} and V_{L}, domains which enables the scFv to form the desired structure for antigen binding. For a review of scFv See Plückthun in The Pharmacology of Monoclonal Antibodies, vol. 113, Rosenburg and Moore eds., Springer-Verlag, New York, pp. 269-315 (1994).

In a further embodiment, antibodies or antibody fragments can be isolated from antibody phage libraries generated using the techniques described in McCafferty et al., Nature, 348: 552-554 (1990). Clackson et al., Nature, 352: 624-628 (1991) and Marks et al., J. Mol. Biol., 222: 581-597 (1991) describe the isolation of murine and human antibodies, respectively, using phage libraries. Subsequent publications describe the production of high affinity (mM range) human antibodies by chain shuffling (Marks et al., Bio/Technology, 16: 779-783 (1992)), as well as combinatorial infection and in vivo recombination as a strategy for constructing very large phage libraries (Waterhouse et al., Nuc. Acids. Res., 21: 2265-2266 (1993)). Libraries of synthetic genes based on the human antibody gene repertoire have also been successfully used for the isolation of therapeutic and diagnostic antibodies (HuCAL library; Knappik et al., J Mol Biol 296(1):57-86 (2000). Thus, these techniques are viable alternatives to traditional monoclonal antibody hybridoma techniques for isolation of monoclonal antibodies. The DNA also may be modified, for example, by substituting the coding sequence for human heavy- and light-chain constant domains in place of the homologous murine sequences (U.S. Pat. No. 4,816,567; Morrison, et al., Proc. Nat. Acad. Sci. USA, 81: 6851 (1984)), or by covalently joining to the immunoglobulin coding sequence all or part of the coding sequence for a non-immunoglobulin polypeptide. Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a Single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as sequiring production of the antibody by any particular method. For example, the monoclonal antibodies to be used in accordance with the present invention may be made by the hybridoma method first described by Kohler et al., Nature, 256: 495 (1975), or may be made by recombinant DNA methods (See, e.g., U.S. Pat. No. 4,816,567).

The monoclonal antibodies may also be isolated from phage antibody libraries using the techniques described in Clackson et al., Nature, 352: 624-628 (1991) and Marks et al., J. Mol. Biol., 222: 581-597 (1991), for example.

The monoclonal antibodies herein specifically include chimeric antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81: 6851-6855 (1984)).

Chimeric antibodies of interest herein include “primatised” antibodies comprising variable domain antigen-binding sequences derived from a non-human primate (e.g. Old World Monkey, such as baboon, rhesus or cynomolgus monkey) and human constant region sequences (U.S. Pat. No. 5,693,780).

Methods for humanising non-human antibodies have been described in the art. Preferably, a humanised antibody has one or more amino acid residues introduced into it from a source which is non-human. These non-human amino acid residues are often referred to as “import” residues, which are typicality taken from an “import” variable domain. Humanisation can be essentially performed following the method of Winter and Co-workers (Jones et al., Nature, 321: 522-525 (1986); Riechmann et al., Nature, 332: 323-327 (1988); Verhoeyen et al., Science, 239: 1534-1536 (1988)), by substituting hypervariable region sequences for the corresponding sequences of a human antibody. Accordingly, such “humanised” antibodies are chimeric antibodies (U.S. Pat. No. 4,816,567) wherein substantially less than an intact human variable domain has been substituted by the corresponding sequence from a non-human species. In practice, humanised antibodies are typically human antibodies in which some hypervariable region residues and possibly some FR residues are substituted by residues from analogous sites in rodent antibodies. The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework region (FR) for the humanised antibody (Sims et al., J. Immunol., 151: 2296 (1993); Chothia et al., J. Mol. Biol., 196: 901 (1987)). Another method uses a particular framework region derived from the consensus sequence of all human antibodies of a particular subgroup of light or heavy chains. The same framework may be used for several different humanised antibodies (Carter et al., Proc. Natl. Acad. Sci. USA, 89:4285 (1992); Presta et al., J. Immunol., 151: 2623 (1993)). It is further important that antibodies be humanized with retention of high affinity for the antigen and other favourable biological properties. To achieve this goal, according to a preferred method, humanised antibodies are prepared by a process of analysis of the parental sequences and various conceptual humanised products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences.

Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

As an alternative to humanisation, human antibodies can be generated. For example, it is now possible to produce transgenic animals (e.g., mice) that are capable, upon immunisation, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90: 2551 (1993); Jakobovits et al., Nature, 362: 255-258 (1993); Bruggermann et al., Year in Immuno., 7: 33 (1993); and U.S. Pat. Nos. 5,591,669; 5,589,369 and 5,545,807.

Alternatively, phage display technology (McCafferty et al., Nature 348: 552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibodies and antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson, Kevin S, and Chiswell, David J., Current Opinion in Structural Biology 3: 564-571 (1993). Several sources of V-gene Segments can be used for phage display. Clackson et al., Nature, 352: 624-628 (1991) isolated a diverse array of anti-oxazolone antibodies from a small random combinatorial library of V genes derived from the Spleens of immunized mice. A repertoire of V genes from unimmunized human donors can be constructed and antibodies to a diverse array of antigens (including self-antigens) can be isolated essentially following the techniques described by Marks et al., J. Mol. Biol. 222581-597 (1991), or Griffith et al., EMBO J. 12: 725-734 (1993). See, also, U.S. Pat. Nos. 5,565,332 and 5,573,905. Human antibodies may also be generated by in vitro activated B cells (see U.S. Pat. Nos. 5,567,610 and 5,229,275).

The term “hypervariable region” when used herein refers to the amino acid residues of an antibody which are responsible for antigen-binding. The hypervariable region comprises amino acid residues from a “complementarily determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196: 901-917 (1987)). “Framework” or “FR” residues are those variable domain residues other than the hypervariable region residues as herein defined.

Amino acid sequence modification(s) of protein or antibody or antibody fragments, as described herein are contemplated. For example, it may be desirable to improve the binding affinity and/or other biological properties of the antibody part. Amino acid sequence variants of the antibody part are prepared by introducing appropriate nucleotide changes into the antibody part nucleic acid, or by peptide synthesis. Such modifications include, for example, deletions from, and/or insertions into and/or Substitutions of, residues within the amino acid sequences of the antibody part. Any combination of deletion, insertion and substitution is made to arrive at the final construct, provided that the final construct possesses the desired characteristics. Therefore, in the case of the heavy or light chain a variation of 1; 2; 3; 4; 5; 6; 7; 8; 9; 10; 11; 12; 13; 14; 15; 16; 17; or 20 amino acids can be executed. In the case of a linker a variation of 1; 2; 3; 4; 5; 6; or 7 can be executed. In case of the linker the variations are much more flexible, because function is simple to create a sufficient space between the functional amino acid sequences. A variation is defined as a deletion, insertion and/or substitution.

The amino acid changes also may alter post-translational processes of the antibody part, such as changing the number or position of glycosylation sites. A useful method for identification of certain residues or regions of the antibody part that are preferred locations for mutagenesis is called “alanine scanning mutagenesis” as described by Cunningham and Welk Science, 244: 1081-1085 (1989). Here, a residue or group of target residues are identified (e.g., charged residues such as Arg, Asp, His, Lys, and Glu) and replaced by a neutral or negatively charged amino acid (most preferably alanine or poly-alanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the Performance of a mutation at a given site, Ala scanning or random mutagenesis is conducted at the target codon or region and the expressed antibody part variants are screened for the desired activity. Amino acid sequence insertions include amino- and/or carboxyl-terminal fusions ranging in length from one residue to Polypeptides containing a hundred or more residues, as well as intrasequence insertions of single or multiple amino acid residues. Examples of terminal insertions include an antibody part with an N-terminal methionyl residue or the antibody part fused to a cytotoxic polypeptide. Other insertional variants of the antibody part molecule include the fusion to the N- or C-terminus of the antibody part of an enzyme, or a polypeptide which increases the serum half-life of the antibody part. Another type of variant is an amino acid substitution variant. These variants have at least one amino acid residue in the antibody part replaced by different residue. The sites of greatest interest for substitutional mutagenesis of antibody parts include the hypervariable regions, but FR alterations are also contemplated. Conservative substitutions are shown in Table 1 under the heading of “preferred substitutions”.

TABLE 1

| Original | | Preferred |
|---|---|---|
| Residue | Exemplary substitution | Substitution |
| Ala | Val; Leu; Ile | Val |
| Arg | Lys, Gln, Asn | Lys |
| Asn | Gln, His, Asp, Lys, Arg | Gln |
| Asp | Glu, Asn | Glu |
| Cys | Ser, Ala | Ser |
| Gln | Asn, Glu | Asn |
| Glu | Asp, Gln, | Asp |
| Gly | Ala | Ala |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, Gln, Asn | Arg |
| Met | Leu, Phe, Ile, | Leu |
| Phe | Leu, Val, Ile, Ala, Tyr | Tyr |
| Pro | Ala | Ala |
| Ser | Thr | Thr |
| Thr | Ser | Ser |
| Trp | Tyr, Phe | Tyr |
| Tyr | Trp, Phe, Thr, Ser | Phe |
| Val | Ile, Leu, Met, Phe, Ala, Norleucine | Leu |

If such substitutions result in a change in biological activity, then more substantial changes, denominated “exemplary substitutions” in Table 1, or as further described below in reference to amino acid classes, may be introduced and the products screened.

Substantial modifications in the biological properties of the antibody part are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: Norleucine, Met, Ala, Val, Leu, Ile;
(2) neutral hydrophilic: Cys, Ser, Thr;
(3) acidic: Asp, Glu;
(4) basic: Asn, Gln, His, Lys, Arg;
(5) residues that influence chain orientation: Gly, Pro; and
(6) aromatic: Trp, Tyr, Phe.

Non-conservative substitutions will entail exchanging a member of one of these classes for another class. Any cysteine residue not involved in maintaining the proper conformation of the antibody part also may be substituted, generally with serine, to improve the oxidative stability of the molecule and prevent aberrant cross linking. Conversely, cysteine bond(s) may be added to the antibody part to improve its stability.

A particularly preferred type of substitutional variant involves substituting one or more hypervariable region residues of a parent antibody. Generally, the resulting variant(s) selected for further development will have improved biological properties relative to the parent antibody from which they are generated. A convenient way for generating such substitutional variants is affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or in additionally, it may be beneficial to analyse a crystal structure of the antigen-antibody complex to identify contact points between the antibody and antigen. Such contact residues and neighbouring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody part alters the original glycosylation pattern of the antibody part. By altering is meant deleting one or more carbohydrate moieties found in the antibody part, and/or adding one or more glycosylation sites that are not present in the antibody part.

Glycosylation of polypeptides, here the antibody parts, is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody part is conveniently accomplished by altering the amino acid sequence such that it contains one or more of the above-described tri-peptide sequences (for N-linked glycosylation sites). The alteration may also be made by the addition of, or substitution by, one or more serine or threonine residues to the sequence of the original antibody part (for O-linked glycosylation sites).

Nucleic acid molecules encoding amino acid sequence variants of the antibody part are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody part.

It may be desirable to modify the antibody part of the invention with respect to effector function, e.g. so as to enhance antigen-dependent cell-mediated cyotoxicity (ADCC) and/or complement dependent cytotoxicity (CDC) of the antibody part. This may be achieved by introducing one or more amino acid substitutions in an Fc region of an antibody part. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumour activity may also be prepared using heterobifunctional cross-linkers as described in Wolff et al. Cancer Research 53:2560-2565 (1993). Alternatively, an antibody can be engineered which has dual Fc regions and may thereby have enhanced complement lysis and ADCC capabilities. See Stevenson et al. Anti-Cancer Drug Design 3:219-230 (1989).

Variations of the antibodies and antibody fragments are defined by variations as mentioned before. That means the variations of special sequences, mentioned in the following list of Table 2, which can be modified be deletion, insertion and/or substitution by the following numbers of amino acids:

TABLE 2

| | Numbers of amino acids, which are deleted, |
|---|---|
| Seq. Id. No. | inserted and/or substituted: |
| 1, 4, 5, 6, 7, 10, | 1, 2, 3, 4 |
| 11, 12, 13, 16, | |
| 17, 18 | |
| 2, 3, 8, 9, 14, 15 | 1, 2, 3, 4, 5, 6, 7, 8 |
| 19, 20, 21, 22, | 1, 2, 3, 4, 5, 6, 7, 8, 9, 10, 11, 12, 13, 14, 15, 16 |
| 23, 24 | 17, 18, 19, 20, 21, 22, 23, 24, 25, 26, 27, 28, |
| | 29, 30 |
| 25, 26, 27, 28, | 1, 2, 3, 4, 5, 6, 7, 8, 9, 10, 11, 12, 13, 14, 15, |
| 29, 30, | 16, 17, 18, 19, 20, 21, 22, 23, 24, 25, 26, 27, |
| | 28, 29, 30, 31, 32, 33, 34, 35, 36, 37, 38, 39, |
| | 40, 41, 42, 43, 44, 45, 46, 47, 48, 49, 50 |

The antibodies or antibody fragments according to the instant invention comprise the variable domain (V) of the complementary determining region (CDR) of a heavy chain (CDR-H, VH) and a light chain (CDR-L, VL), whereby the heavy chain (CDR-H) is selected from the group of Seq. ID NO. 19, Seq. ID NO. 21 and Seq. ID NO. 23; and the light chain (CDR-L) is selected from the group of Seq. ID NO. 20, Seq. ID NO. 22 and Seq. ID NO. 24.

Especially the instant invention concerns antibodies or antibody fragments, which comprise the constant domain (C) of the complementary determining region (CDR) of a heavy chain (CDR-H, CH) and a light chain (CDR-L, CL), wherein the heavy chain (CDR-H) is selected from the group of Seq. ID NO. 25 and the light chain (CDR-L) is selected from the group of Seq. ID NO. 26, or the heavy chain (CDR-H) is selected from the group of Seq. ID NO. 27 and the light chain (CDR-L) is selected from the group of Seq. ID NO. 28, or the heavy chain (CDR-H) is selected from the group of Seq. ID NO. 29 and the light chain (CDR-L) is selected from the group of Seq. ID NO. 30.

The inventive antibodies or antibody fragments may further comprise the constant domain (C) of the complementary determining region (CDR) of a heavy chain (CDR-H, CH) and a light chain (CDR-L, CL), wherein the heavy chain (CDR-H) is selected, for example, from the group of Seq. ID NO. 25, Seq. ID NO. 27 and Seq. ID NO. 29; and the light chain (CDR-L) is, for example, selected from the group of Seq. ID NO. 26, Seq. ID NO. 28 and Seq. ID NO. 30.

The antibodies or antibody fragments according to the instant invention may comprise the complementary determining region (CDR) of a heavy chain (CDR-H) and a light chain (CDR-L) on the Fab fragment. The heavy chain (CDR-H, FabH) may be selected from the group of Seq. ID NO. 32, Seq. ID NO. 34, Seq. ID NO. 36, Seq. ID NO. 38, Seq. ID NO. 40, Seq. ID NO. 42, Seq. ID NO. 44, Seq. ID NO. 46, Seq. ID NO. 48, Seq. ID NO. 50, Seq. ID NO. 52, Seq. ID NO. 54, Seq. ID NO. 56, Seq. ID NO. 58, Seq. ID NO. 60, Seq. ID NO. 62, Seq. ID NO. 64, Seq. ID NO. 66, Seq. ID NO. 68, Seq. ID NO. 70, Seq. ID NO. 72, Seq. ID NO. 74, Seq. ID NO. 76, Seq. ID NO. 78, Seq. ID NO. 80, Seq. ID NO. 82, Seq. ID NO. 84, Seq. ID NO. 86, Seq. ID NO. 88, Seq. ID NO. 90 and Seq. ID NO. 92; and the light chain (CDR-L, FabL) may be selected from the group of Seq. ID NO. 31, Seq. ID NO. 33, Seq. ID NO. 35, Seq. ID NO. 37, Seq. ID NO. 39, Seq. ID NO. 41, Seq. ID NO. 43, Seq. ID NO. 45, Seq. ID NO. 47, Seq. ID NO. 49, Seq. ID NO. 51, Seq. ID NO. 53, Seq. ID NO. 55, Seq. ID NO. 57, Seq. ID NO. 59, Seq. ID NO. 61, Seq. ID NO. 63, Seq. ID NO. 65, Seq. ID NO. 67, Seq. ID NO. 69, Seq. ID NO. 71, Seq. ID NO. 73, Seq. ID NO. 75, Seq. ID NO. 77, Seq. ID NO. 79, Seq. ID NO. 81, Seq. ID NO. 83, Seq. ID NO. 85, Seq. ID. NO. 87, Seq. ID NO. 89 and Seq. ID NO. 91.

In another aspect of the instant invention, an antibody may comprise the heavy chain (IgG4 H) wherein the sequences are selected from the group of Seq. ID NO. 94, Seq. ID NO. 96, Seq. ID NO. 98, Seq. ID NO. 100, Seq. ID NO. 102, Seq. ID NO. 104, Seq. ID NO. 106, Seq. ID NO. 108, Seq. ID NO. 110, Seq. ID NO. 112, Seq. ID NO. 114 and Seq. ID NO. 116; and for the light chain (IgG4 L), the sequences are selected from the group of Seq. ID NO. 93, Seq. ID NO. 95, Seq. ID NO. 97, Seq. ID NO. 99, Seq. ID NO. 101, Seq. ID NO. 103, Seq. ID NO. 105, Seq. ID NO. 107, Seq. ID NO. 109, Seq. ID NO. 111, Seq. ID NO. 113 and Seq. ID NO. 115.

A preferred antibody according to the instant invention comprises the heavy chain (IgG1 H) Seq. ID NO. 118 and the light chain (IgG1 L) Seq. ID NO. 117.

The havy and light chains within the antibodies can be located in such a way that the heavy chain is at the C-terminus and the light chain is at the N-terminus or the heavy chain is at the N-terminus and the light chain is at the C-terminus. The antibodies may be monovalent or polyvalent.

The inventive antibodies and antibody fragments exhibit pharmacological activity and are, therefore, useful as pharmaceuticals. In particular, the antibodies and antibody fragments show pharmacological activity in a number of pathological or disease states in connection with a cancer, especially solid cancer, more especially breast cancer.

The antibodies and antibody fragments of the invention may also be administered, alone or in combination (simultaneously or in doses at different times).

For the treatment of such conditions, the appropriated dosage will, of course, vary depending upon, for example, the host, the mode of administration and the nature and severity of the condition being treated.

The antibodies and antibody fragments of the present invention may be administered to a patient in need of treatment via any suitable route, usually by invention into the bloodstream and/or directly into the site to be treated, e.g. tumour. The precise dose will depend upon a number of factors, the route of treatment, the size and location of the area to be treated (e.g. tumour), the precise nature of the antibody and antibody fragment (e.g. Fab′, F(ab′)2, scFv molecule), and the nature of any detectable label or other molecule attached to the antibody. A typical antibody fragment dose will be in the range 5-50 mg/kg. This is a dose for a single treatment of an adult patient, which may be proportionally adjusted for children and infants, and also adjusted for other antibody formats in proportion to molecular weight. Treatments may be repeated at daily, twice-weekly, weekly or monthly intervals, at the discretion of the physician.

The present invention also provides pharmaceutical compositions comprising the antibodies and/or antibody fragments of the invention (specifically recognising the EphB4 receptor) in association with at least one pharmaceutically acceptable carrier or diluent. Such composition may be manufactured in conventional manner. Pharmaceutically acceptable carriers or diluents are described in Remington's Pharmaceutical Science, 15^{th} ed. Mack Publishing Company, Easton Pa. (1980).

A further embodiment of the instant invention is the use of the antibodies or antibody fragments as a medicament. The invention thus further concerns
(i) the use of the pharmaceutical composition of the antibodies and antibody fragments of the invention for manufacture of a medicament for treatment of cancer especially breast cancer;
(ii) a method of treatment of cancer especially breast cancer;
   which method comprises an administration of a pharmaceutical composition of the antibodies and antibody fragments of the invention, wherein the amount of combination suppresses the disease and wherein the pharmaceutical composition of the combination is given to a patient, who is in need thereof;
(iii) a pharmaceutical composition of the combination of the invention for treatment of cancer especially breast cancer,
   which treatment comprises a pharmaceutical composition of the invention and at least one pharmaceutically acceptable carrier or diluent.

The antibodies or antibody fragments may further be used in combination with a cancer active compound.

A further embodiment of the instant invention are those nucleic acids, such as DNA's, which encode an antibody or antibody fragment of the instant invention.

The instant invention further concerns those expression vectors host cells, which can be used for the transformation and transfection these nucleic acids, as well as the method for producing the inventive antibodies or antibody fragments, using a host cell by culturing the host cell in a suitable medium under conditions wherein the nucleic acids are encoded and the antibodies or antibody fragments are expressed and are recovered from the host cell or the medium.

The preferred method of administration is the injection of the antibody fragment, the parenteral administration.

Preferred antibodies and antibody fragments of the invention have an affinity (K_{D}) of at least 100 nM, more preferably of at least 10 nM human and/or murine EphB4 and can bind both native and recombinant EphB4. The K_{D} of antibodies and antibody fragments binding to EphB4 can be assayed using any method known in the art, including technologies such as real-time Bimolecular Interaction Analysis Interaction Analysis (BIA) (Sjolander & Urbaniczky, Anal. Chem. 63, 2338-45, 1991, and Szabo et al., Curr. Opin. Struct. Biol. 5, 699-705, 1995). BIA can be used to study biospecific interactions in real time without labelling any of the interactants (e.g., BIACORE™). Changes in the optical phenomenon surface plasmon resonance (SPR) can be used as an indication of real-time reactions between biological molecules.

In a BIACORE™ assay, preferred antibodies and antibody fragments of the invention specifically bind to human EphB4 with a K_{D} of about 1 nM to about 40 nM. More preferred human antibodies specifically bind to human EphB4 with a K_{D} of equal or less than 1, 1.1, 1.3, 1.8, 2, 3, 4, 5, 6, 7, 8, 10, 12, 16, 20, 25, 30, 30, 35, or 40 nM.

A human antibody can be produced using chemical methods to synthesize its amino acid sequence, such as by direct peptide synthesis using solid-phase techniques (Merrifield, J. Am. Chem. Soc. 85, 2149-54, 1963; Roberge et al., Science 269, 202-04, 1995). Protein synthesis can be performed using manual techniques or by automation. Automated synthesis can be achieved, for example, using Applied Biosystems 431A Peptide Synthesizer (Perkin Elmer). Optionally, fragments of a human antibody can be separately synthesized and combined using chemical methods to produce a full-length molecule.

The newly synthesized molecules can be substantially purified by preparative high performance liquid chromatography (e.g., Creighton, proteins: structures and molecular principles, W H Freeman and Co., New York, N.Y., 1983). The composition of a synthetic polypeptide can be confirmed by amino acid analysis or sequencing (e.g., using Edman degradation).

Preferably, however, antibodies of the invention are produced recombinantly by a host cell which has been transfected with a human antibody-encoding expression construct, as described below. The host cell is cultured in conditions under which the antibodies are expressed. Antibodies can be purified from the host cell and can be separated from other compounds that normally associate with the antibodies in the cell (such as proteins, carbohydrates, or lipids) using methods well known in the art. Such methods include, but are not limited to, size exclusion chromatography, ammonium sulphate fractionation, ion exchange chromatography, affinity chromatography, and preparative gel electrophoresis. Alternatively, antibodies can be produced containing a signal sequence which causes their secretion into the culture medium.

A preparation of purified antibodies and antibody fragments of the invention is at least 80% pure (80% antibody, 20% other proteins), preferably, the preparations are 90%, 95%, or 99% pure. Purity of the preparations can be assessed by any means known in the art, such as SDS-polyacrylamide gel electrophoresis. A preparation of purified antibodies and antibody fragments of the invention can contain more than one type of EphB4 antibodies and antibody fragments. The invention provides nucleic acid molecules, e.g. DNA molecules encoding all or a portion of EphB4 antibodies of the invention. The nucleic acid molecules may encode a light chain and/or a heavy chain. The nucleic acid can be used, for example, to produce quantities of the antibodies for therapeutic or diagnostic use.

A nucleic acid molecule of the invention can be inserted into an expression vector which contains the necessary elements for the transcription and translation of the inserted coding sequence, thereby forming an expression construct. Methods that are well known to those skilled in the art can be used to make expression constructs containing sequences encoding antibodies of the invention or portions thereof. These methods include in vitro recombinant DNA techniques, synthetic techniques, and in vivo genetic recombination. Such techniques are described, for example, in Sambrook et al., molecular cloning: a laboratory manual, 2d ed., 1989, and in Ausubel et al., current protocols in molecular biology, John Wiley & Sons, New York, N.Y., 1995.

A variety of expression vector/host systems can be utilized to contain and express a nucleic acid molecule encoding a human antibody of the invention. These include, but are not limited to, microorganisms, such as bacteria transformed with recombinant bacteriophage, plasmid, or cosmid DNA expression vectors or yeast transformed with yeast expression vectors. Insect cell systems infected with virus expression vectors (e.g., baculovirus), plant cell systems transformed which, e.g., cauliflower mosaic virus, tobacco mosaic virus, or with a bacterial expression vector (e.g., Ti or pBR322 plasmids), or animal cell systems also can be used.

Control elements or regulatory sequences present in an expression construct are those non-translated regions (e.g., enhancers, promoters, 5′ and 3′ untranslated regions) which interact with host cellular proteins to carry out transcription and translation. Such elements can vary in their strength and specificity. Depending on the vector system and host used, any number of suitable transcription and translation elements, including constitutive and inducible promoters, can be used. For example, when cloning in bacterial systems, inducible promoters such as the hybrid lacZ promoter of the BLUESCRIPT® phagemid (Stratagene, LaJolla, Calif.), pSPORT1 plasmid (Life Technologies), and the like can be used. The baculovirus polyhedrin promoter can be used in insect cells. Promoters or enhancers derived from the genomes of plant cells (e.g., heat shock, RUBISCO, and storage protein genes) or from plant viruses (e.g., viral promoters or leader sequences) can be included in an expression construct. In mammalian cell systems, promoters from mammalian genes or from mammalian viruses are preferable. To generate a cell line which contains multiple copies of a nucleic acid molecule of the invention, constructs based an SV40 or EBV can be used with an appropriate selectable marker.

Large scale production of EphB4 antibodies and antibody fragments can be carried out using methods such as those described in Wurm et al., Ann. N.Y. Acad. Sci. 782, 70-78, 1996, and Kim et al., Biotechnol. Bioengineer 58, 73-84, 1998.

The invention also provides methods of using EphB4 antibodies and antibody fragments to modulate, e.g. to inhibit or prevent the binding of EphrinB2 to EphB4. Such methods can be used therapeutically, as described below, or in a research setting, for example in drug screening.

Further, the invention provides therapeutic methods of inhibiting angiogenesis and treating angiogenesis-associated diseases or disorders including, but not limited to, angiogenesis-dependent cancers (e.g., solid tumours, leukemias, tumour metastases, benign tumours such as hemangiomas, acoustic neuromas, neurofibromas, trachomas, and pyogenic granulomas), inflammatory disorders (e.g., immune and non-immune inflammation), chronic articular rheumatism and psoriasis, ocular angiogenic diseases, Osler-Webber Syndrome, myocardial angiogenesis, plaque neovascularization, telangiectasia, hemophiliac joints, angiofibroma, and wounds. The methods involve administering a therapeutic amount of a pharmaceutical composition of the invention (described below) to an individual who needs such therapy. Pharmaceutical compositions of the invention can be administered by any number of routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means. Pharmaceutical compositions of the invention can be administered to a patient alone, or in combination with further medicaments (in the same or in a different composition), e.g. with further anti tumour-agents such as chemotherapeutic agents.

Pharmaceutical compositions of the invention comprise EphB4 antibody or antibody fragments or a nucleic acid molecule of the invention and a pharmaceutically acceptable carrier. The pharmaceutically acceptable carrier preferably is non-pyrogenic. The compositions can be administered alone or in combination with at least one other agent, such as stabilizing compound, which can be administered in any sterile, biocompatible pharmaceutical carrier, including, but not limited to, saline, buffered saline, dextrose, and water. A variety of aqueous carriers may be employed, e.g., 0.4% saline, 0.3% glycine, and the like. These solutions are sterile and generally free of particulate matter. These solutions may be sterilized by conventional, well known sterilization techniques (e.g., filtration). The compositions may contain pharmaceutically acceptable auxiliary substances as required to approximate physiological conditions such as pH adjusting and buffering agents, etc. The active agent in pharmaceutical compositions is an EphB4 antibody fragment. A given effective amount will vary from condition to condition and in certain instances may vary with the severity of the condition being treated and the patient's susceptibility to treatment. See U.S. Pat. No. 5,851,525. If desired, a pharmaceutical composition can comprise more than one type of antibody, for example with different K_{D} for EphB4 binding or with different IC₅₀s for inhibiting an EphB4 function, can be included in a pharmaceutical composition.

After pharmaceutical compositions have been prepared, they can be placed in an appropriate container and labelled for treatment of an indicated condition. Such Labelling would include amount, frequency, and method of administration.

A “therapeutically effective dose” is an amount of antibody or antibody fragment which reduces the severity of at least one symptom of the disorder being treated relative to the severity of the symptom which occurs in the absence of the therapeutically effective dose. Determination of a therapeutically effective dose is well within the capability of those skilled in the art. For example, a therapeutically effective dose can be estimated initially either in cell culture assays or in animal models, usually rats, mice, rabbits, dogs, or pigs. An animal model also can be used to determine the appropriate concentration range and route of administration. Such information can then be used to determine useful doses and routes for administration in humans.

Therapeutic efficacy and toxicity, e.g., ED₅₀ (the dose therapeutically effective in 50% of the population) and LD₅₀ (the dose lethal to 50% of the population) of a human antibody, can be determined by standard pharmaceutical procedures in cell cultures or experimental animals. The dose ratio of toxic to therapeutic effects is the therapeutic index, and it can be expressed as the ratio LD₅₀/ED₅₀.

Pharmaceutical compositions which exhibit large therapeutic indices are preferred. The dosage contained in pharmaceutical compositions of the invention preferably is within a range of circulating concentrations which include the ED₅₀ with little or no toxicity. The dosage varies within this range depending upon the dosage form employed, sensitivity of the patient, and the route of administration.

The exact dosage will be determined by the practitioner, in light of factors related to the patient who requires treatment. Dosage and administration are adjusted to provide sufficient levels of the human antibody or to maintain the desired effect. Factors which can be taken into account include the severity of the disease state; general health of the subject; age, weight, and gender of the subject; diet; time and frequency of administration; drug combination(s); reaction sensitivities; and tolerance or response to therapy. Long-acting pharmaceutical compositions can be administered every 3 to 4 days, every week, or once every two weeks depending an the half-life and clearance rate of the particular formulation.

Effective in vivo dosages of a human antibody or antibody fragment are in the range of about 5 mg to about 50 mg/kg, about 100 mg to about 500 mg/kg of patient body weight, and about 200 to about 250 mg/kg of patient body weight. For administration of nucleic acid molecules encoding the antibodies and antibody fragments, effective in vivo dosages are in the range of about 100 ng to about 200 ng, 500 ng to about 50 mg, about 1 mg to about 2 mg, about 5 mg to about 500 mg, and about 20 mg to about 100 mg of DNA.

Those skilled in the art will understand that various changes in form and details may be made to the invention defined by the following examples and the appended claims. Such equivalents are encompassed within the scope of the claims.

### DESCRIPTION OF THE FIGURES

FIG. 1 shows the inhibition of EphrinB2 binding to EphB4. EphrinB2 is inhibited to EphB4 but not to EphB3 (e.g. MOR03640). The specificity of anti-EphB4 Fab fragments is shown.

FIG. 2 shows the Inhibition of the ligand-induced phosphorylation of EphB4.

FIG. 3 shows the EphB4 Phosphorylation by dimeric anti-EphB4 Fabs.

FIG. 4 shows the FACS analysis of MCF-7 breast cancer cell line with anti-EphB4 Fabs.

Top panel A: unstained cells.

Middle panel B: stained with anti-EphB4 Fabs and secondary antibody (anti-lyso=anti-lysozyme negative control Fab).

Bottom panel C: secondary antibody only.

FIG. 5 shows the anti-EphB4 Fab precipitate human EphB4 receptor from CHO-EphB4 cells.

FIG. 6 shows the Sequence of the anti EphB4 Fab fragments MOR03640; MOR03639 and MOR03641, each heavy and light chain. The CDRs are written in bold, the constant parts are underlined, the purification peptide tag (poly His) follows the constant part of MOR03640_MH heavy chain and is not underlined.

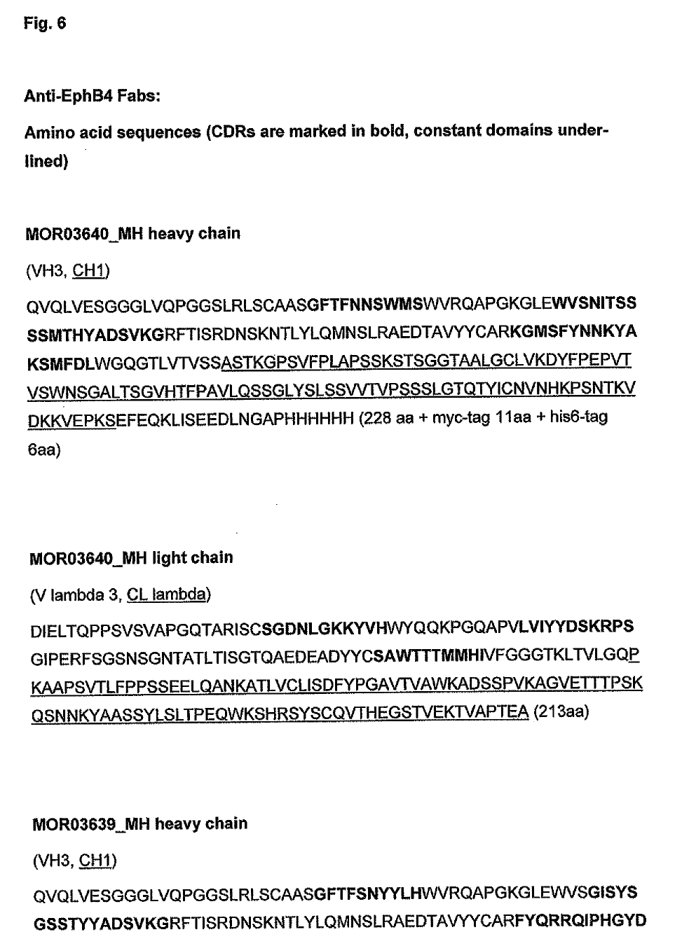

FIG. 7 shows the specificity of Fabs for EphB4 and cross-reactivity to both human and murine EphB4 in ELISA.

FIG. 8 shows the inhibition of EphrinB2 binding to EphB4. Binding of EphrinB2 to EphB4, but not to EphB3 is inhibited by anti-EphB4 specific Fab's (e.g. MOR03640).

FIG. 9 shows that anti-EphB4 Fab MOR 03640 binds to hEphB4-transfected CHO cells.

It should be noted that with increasing numbers of passages, the transfected CHO cells lose hEphB4 which is reflected in the incomplete shift of the anti-EphB4-labeled cells.

FIG. 10 shows the inhibition of the mEphrinB2 binding to mEphB4 by anti-EphB4 Fabs.

FIG. 11 shows the inhibition of the mEphrinB2 binding to hEphB4 by anti-EphB4 Fabs.

FIG. 12 shows the inhibition of the mEphrinB2 binding to CHO-hEphB4 cells. Note: The anti-EphB4 Fabs inhibit EphrinB2 binding to hEphB4-transfected CHO cells with increasing concentration.

FIG. 13 (part 1 and part 2) shows the specificity/cross-reactivity ELISA on human and mouse EphB4 and related Eph receptors.

FIG. 14x show the EC₅₀ determination of matured Fab on CHO-EphB4 cells in FACS

FIG. 14A-1 shows exemplarily the EC₅₀ curves of a set of matured Fabs from the MOR03641 pool.

FIG. 14 A-2 shows exemplarily the EC₅₀ curves of a set of matured Fabs from the MOR03641 pool.

FIG. 14 B-1 shows exemplarily the EC₅₀ curves of a set of matured Fabs from the MOR07310 pool.

FIG. 14 B-2 shows exemplarily the EC₅₀ curves of a set of matured Fabs from the MOR07310 pool

FIG. 15 (part 1 and part 2) shows the inhibition of EphB4 receptor phosphorylation, Fab-format (CHO-EphB4)

FIG. 16x show the EC₅₀ determination of 12 final binders in IgG4-Pro format on CHO-EphB4 cells in FACS

FIG. 16 A shows the EC₅₀ data of selected matured IgGs

FIG. 16 B shows the EC₅₀ data of selected matured IgGs and cross-cloned IgG

FIG. 16 C shows the EC₅₀ data of cross-cloned IgGs

FIG. 17 x show the IC₅₀ determination of 12 final binders in IgG4-Pro format on CHO-EphB4 cells in FACS.

FIG. 17 A shows the IC₅₀ data of selected matured IgGs.

FIG. 17 B shows the IC₅₀ data of selected matured IgGs and cross-cloned IgGs.

FIG. 17 C shows the C: IC₅₀ data of cross-cloned IgGs.

FIG. 18 (part 1 and part 2) shows the inhibition of EphB4 receptor phosphorylation, IgG4-pro format (CHO-EphB4).

FIG. 19 x show the inhibition of cell adhesion by anti-EphB4-IgG4-Pros.

FIG. 19 A shows the testing of 12 IgG4-Pros in adhesion assay on different cell lines Best results were seen for the cross cloned IgGs MOR07953 and MOR07954 (derived from MOR07310 parental clone) on PC3-MM2 prostate cancer cells.

An inhibition of more than 80% at a concentration of 50 μg/ml was observed.

From the matured constructs only MOR07720 (derived from MOR07310) showed an inhibition of more than 50%.

FIG. 19 B shows the dose response curve for the inhibition of PC3-MM2 adhesion by MOR07953 and MOR07954.

FIG. 19 C shows the inhibition of MDA-MB435 cell adhesion by anti-EphB4-IgG4-Pros. MOR07953 and MOR07954 showed best effects (but in lower extent compared to the effects on PC3-MM2 cells). MOR03207 was again used as isotype control.

FIG. 20 x show the results of EphB4 antibodies and Fabs in migration assay on PC3-MM2 and MDA-MB 435 cells.

FIG. 20 A shows the testing of the migration of PC3- and MDA-MB 435s-cells without antibodies. The migration was determined on uncoated and ephrin B2 coated plates. bFGF was used as stimulus both cell types migrated under chosen condition. Results: Coating with ephrin B2 improved migration ability, coating concentration of ephrin B2 is critical:

1.) cell dependent
2.) high concentration might result in detachment of cells

FIG. 20 B shows the results for migration of MDA-MB 435 cells.

FIG. 20 C shows the results for migration of PC3-MM2 cells

### EXAMPLES

### Example 1

### Isolation of EphB4-Specific Fab Antibody Fragments

Synthetic Fab antibody fragments were isolated from the HuCAL GOLD® antibody library (HuCAL®; Knappik et al., J. Mol. Biol. 296, 57-86, 2000). Selection strategy and screening on both recombinant antigens and transfected cells ensured isolation and identification of Fabs cross-reactive to both the murine and the human EphB4 extracellular domain. Three Fab antibody fragments were identified. The Fab fragments comprise VH-CH1 and VL-CL nucleic acids.

Specificity for EphB4 in ELISA: Wells of a 96-well maxisorp-plate were coated overnight with 100 μL recombinant human EphB4 (hEphB4)-, recombinant mouse EphB3 (mEphB3)- or recombinant mouse EphA4 (mEphA4)-Fc fusion protein at a final concentration of 2.5 μg/mL in PBS. Anti-EphB4 Fab fragments (purified, MH-tagged) were added (100 μL/well, 100 μg/mL final concentration in chemiblock) to respective wells and incubated for 1 h at RT. Then 100 μl mEphrinB2-Fc fusion protein either spiked with the respective anti-EphB4 Fab or with PBS was added (25 ng/ml mEphrinB2+/−100 μg/ml Fab diluted in chemiblock) and incubated for max. 20 min at RT. The amount of bound EphrinB2 was detected using biotinylated goat anti-EphrinB2 antibody (0.05 μg/ml final concentration diluted in chemiblock, 1 h at RT), AP-conjugated streptavidin and AttoPhos substrate. Fluorescence was measured (RFU) at 535 nm (430 nm excitation).

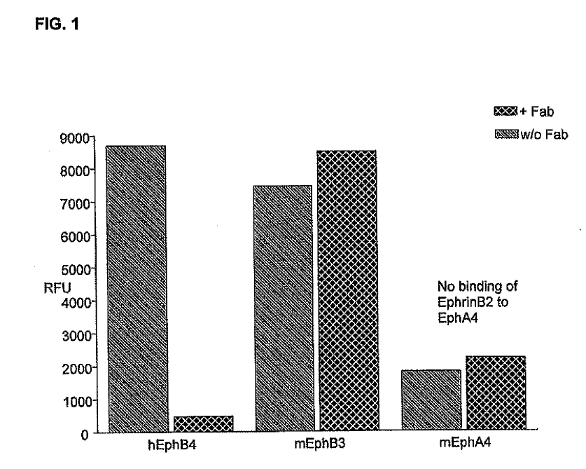

The results are shown in FIG. 1.

Binding of the ligand EphrinB2 to its receptor can potently be blocked by the anti-EphB4 Fab MOR03640: a high fluorescence signal (RFU) indicates unhindered ligand-receptor, binding as is the case for mEphrinB2 binding to mEphB3. On the other hand binding of the ligand mEphrinB2 to the receptor hEphB4 is clearly prevented by addition of the anti-EphB4 Fab indicating that this Fab show specific inhibition of the mEphrinB2-hEphB4 interaction.

### Example 2

### Affinity of Anti-EphB4 Fab's

The affinity of the antibody fragments was determined using BIACORE™ analysis and cell ELISA. The Biacore chip was coated with recombinant human or murine EphB4-Fc fusion protein.

The results are shown in Table 3.

TABLE 3

K_{D} (nM) for EphB4

| Antibody | Human | Murine |
|---|---|---|
| MOR03639 | 13 ± 3 | 43 ± 4 |
| MOR03640 | 1.8 ± 0.7 | 4 ±_0.4 |
| MOR03641 | 35 ± 5 | 151 ± 21 |

The Fab's show nanomolar affinities for both the murine and the human EphB4 receptor (extracellular domains). MOR03640 shows the highest affinities for both receptors, with very similar affinities for the two different species.

IC₅₀ values were determined using recombinant antigen (murine and human EphB4) in an ELISA and on transfected cells in a FACS analysis (human EphB4 receptor). The results are shown in Table 4.

TABLE 4

| | IC₅₀ (μg/mL) inhibition | | IC₅₀ (μg/mL) inhibition |
|---|---|---|---|
| | human EphB4 | | murine EphB4 |
| Antibody | ELISA | FACS | ELISA |
| MOR03639 | 65 ± 5 | 2.8 | 22 ± 8 |
| MOR03640 | 33 ± 9 | 2.1 | 117 ± 21 |
| MOR03641 | 5 ± 0.8 | 4.4 | 24 ± 0.2 |

Binding of (a fixed concentration of) the ligand EphrinB2-Fc to immobilized EphB4 receptor on an ELISA plate or on transfected cells is titrated with anti-EphB4 Fab. The IC₅₀ is determined as the amount of Fab at which the binding of EphrinB2 is inhibited by 50%.

### Example 3

### Inhibition of hEphB4 Phosphorylation by Monomeric Fab

CHO cells were transfected with human EphB4 containing a myc tag. Transfected cells were seeded into 96-well microtiter plates and incubated at 37° C., 5% CO₂ for 24 hours. Monomeric EphB4 Fabs were added to the wells in different concentrations and incubated for 30 minutes, then EphrinB2-Fc was added to the wells in a final concentration of 250 ng/mL. After a further incubation for 30 minutes, the cells were washed once with PBS and then lysed. EphB4 in the lysates was captured via its myc-tag. Phosphorylation was detected with a peroxidase-coupled anti-phospho-tyrosine antibody.

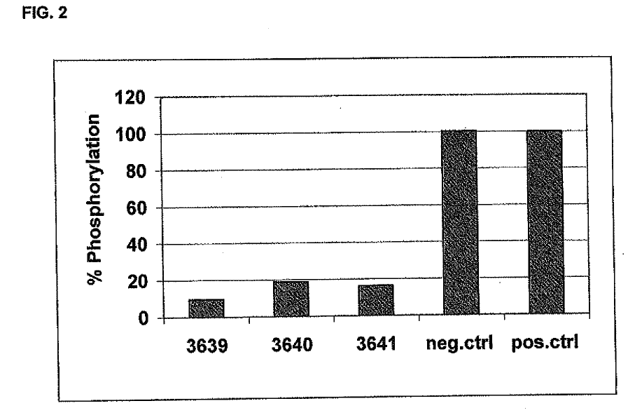

The results are shown in FIG. 2. The ligand-induced phosphorylation (activation) of the EphB4 receptor on the transfected CHO cells is measured via a peroxidase-coupled anti-phospho-tyrosine antibody. Positive control: 100% phosphorylation of the receptor by EphrinB2 alone, negative control: EphrinB2 plus an irrelevant Fab antibody; this Fab does not bind to the EphB4 receptor, thus does not inhibit binding of the EphrinB2 ligand to the receptor and therefore also leads to 100% phosphorylation of the receptor. The anti-EphB4 Fabs 3639, 3640 and 3541 block binding of the EphrinB2 ligand to the EphB4 receptor, resulting in strongly reduced phosphorylation of the receptor.

### Example 4

### Phosphorylation of EphB4 by Dimeric Fab

The assay was performed as described above, with the following modifications: dimeric Fab fragments (20 μg/mL) were pre-incubated with an anti-myc tag antibody at 60 or 120 μg/mL at room temperature for 30 minutes before the mixture was added to the cells. As a positive control, one set of wells was incubated with EphrinB2-Fc only at a concentration of 250 ng/mL.

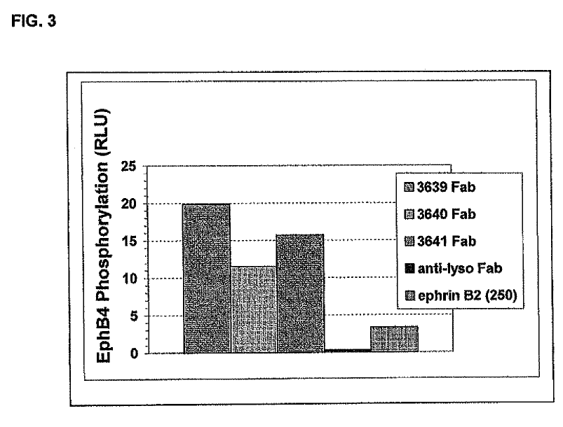

The results are shown in FIG. 3.

Dimeric Fab antibody fragments mimic the EphrinB2 ligand, leading to a strong phosphorylation of the EphB4 receptor.

### Example 5

### MCF-7 Breast Cancer Cell Line is EphB4 Positive

FACS analysis was used to confirm that the MCF-7 breast cancer cell line is highly positive for EphB4 expression. EphB4 Fabs were incubated at a concentration of 10 μg/mL with 10⁶ MCF-7 cells at 4° C. for 1 hour. After washing with PBS, the cells were incubated with FITC-labelled secondary antibody. After a final PBS wash, the cell-associated fluorescence was measured using a Becton Dickinson Calibur Fluorescence activated cell sorter.

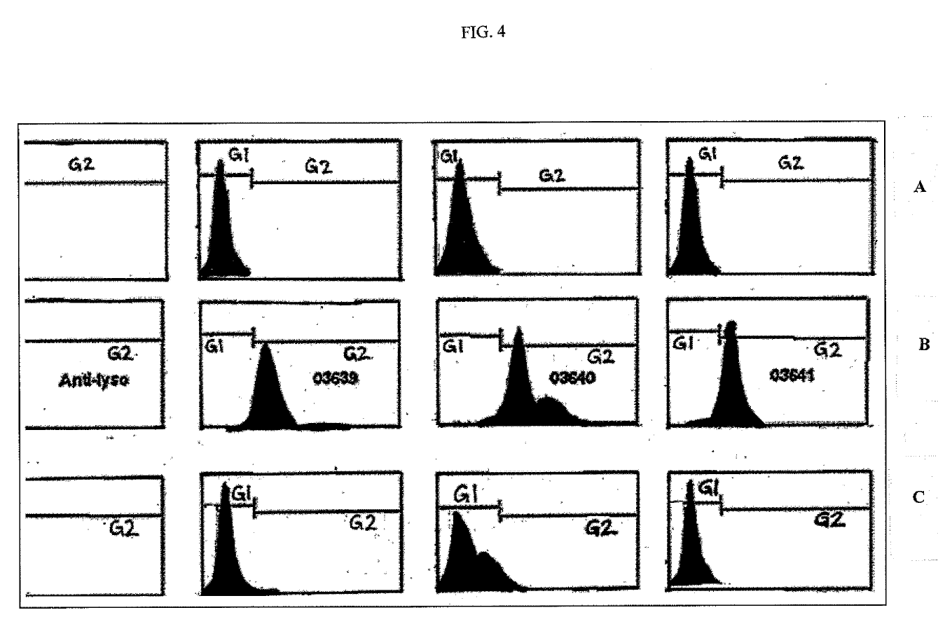

The results are shown in FIG. 4. Each column is the result of at least two independent experiments. The top panel shows unlabeled cells, the bottom panel cells incubated which the secondary antibody (anti-myc tag antibody) only. The middle panel Shows cells incubated with both first (negative control: anti-lysozyme Fab, anti-EphB4 Fabs 03639, 03640, 03641) and secondary antibodies. Upon incubation which the anti-EphB4 Fabs, the MCF-7 cells show a shift, indicating recognition of the cell-surface EphB4 receptor by the Fabs. The negative control Fab does not stain the cells.

### Example 6

### Anti-EphB4 Fab Precipitate Human EphB4 Receptor from CHO-EphB4 Cells

### Method

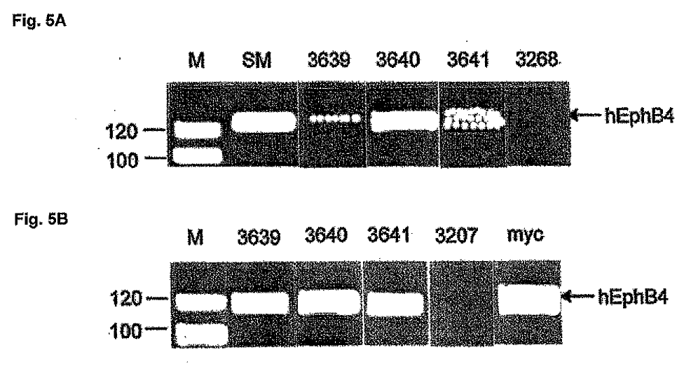

CHO-EphB4 cells (Sturz et al. 2003) were lysed in lysis buffer (50 mM Hepes pH 7.2, 150 mM NaCl, MgCl₂ 1 mM, 10% Glycerine (v/v), 1.5% Triton X-100 (v/v), 2% phosphatase inhibitor cocktail 2 (v/v, Sigma), 4% protease inhibitor cocktail complete (v/v, Roche) and the lysate was cleared by centrifugation and pre-absorbed to protein A agarose beads (4 fast flow; Sigma). For immunoprecipitation 5 μg of antibodies (dissolved to 0.5 μg/μl in PBS) were added to 100 μg (1 μg/μL) of cell lysate and incubated over night at 4° C. 10 μg antibodies against human IgG (Jackson Immuno Research) pre-adsorbed to Protein A beads were added and incubated for 1 h at 4° C. to collect the immunocomplexes. In the case of anti-myc antibody (mAb 9E10, Roche) only Protein A beads were added. Protein A beads were pelleted by centrifugation and washed for three times with 1 mL of lysis buffer and once with 1 mL of PBS. Bound material was removed with SDS gel loading buffer for 15 min at 70° C. and separated an 4-12% SDS polyacrylamide gels in Tris/tricine buffer (Invitrogen). Proteins were transferred to nitrocellulose membranes and EphB4 was detected by Western blot using mouse monoclonal anti-myc antibodies and anti mouse IgG antibodies coupled with horseradish peroxidase (Amersham Pharmacia Biotech). The blot was developed using chemiluminescence. See FIG. 5A.

EphB4 was immunoprecipitated from CHO-EphB4 cell lysate as described in the methods section using monomeric EphB4 Fabs 3639, 3640 and 3641. An unrelated Fab was used as negative control (3268). M: Marker; SM: starting material. See FIG. 5 B.

EphB4 was immunoprecipitated from CHO-EphB4 cell lysate as described in the methods section using dimeric EphB4 Fabs 3639, 3640 and 3641. An unrelated Fab was used as negative control (3207). As a positive control, the myc-tagged EphB4 was immunoprecipitated with a mAb against the myc epitope (myc). M: Marker.

### Example 7

### Cross-Reactivity of Fabs for Human and Murine EphB4 and Specificity of Fabs for EphB4, not Related Ephs

A 96-well plate was coated with recombinant mEphB1, -2, -3, -4, -6, mEphA4 and hEphB4 in PBS o/n at 4° C. (5 μg/mL; 100 μL/well). The next day, the antigen was discarded and the plate blocked with 3% BSA/PBS (300 μl/well) for 2 hrs at room temperature (RT). Blocking solution was discarded and anti-EphB4 Fabs were added to the wells at a concentration of 20 μg/mL in 1% BSA/PBS and incubated for 1 hr at RT. Fabs were discarded and the plate was washed 3× with PBS. Anti-human F(ab′)₂—HRP was added at a concentration recommended by the manufacturer in 1% BSA/PBS and incubated for 1 hr at RT. The plate was washed 3× with PBS and 100 μL/well chemifluorescent substrate was added. After development, fluorescence was measured in a fluorescence reader.

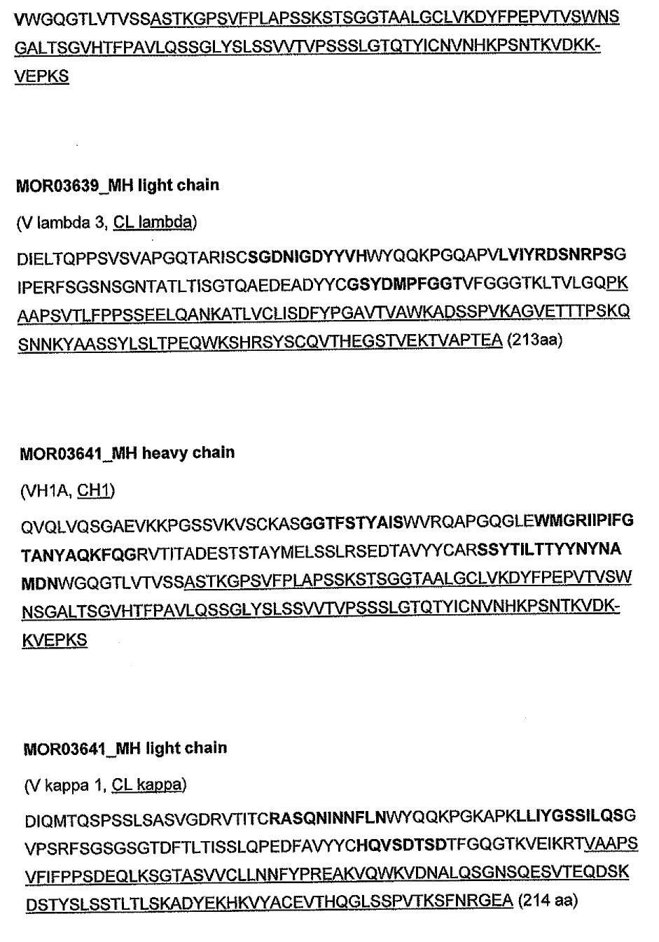

The results are shown in FIG. 7. The anti-EphB4 Fabs show specificity for EphB4 and are cross-reactive to both human and murine EphB4.

### Example 8

### Inhibition of mEphrinB2 Binding to hEphB4, but not Other Eph Receptors

A 96-well plate was coated with recombinant mEphB3, mEphA4 and hEphB4 in PBS o/n at 4° C. (5 μg/mL; 100 μL/well). The next day, the antigen was discarded and the plate blocked with 3% BSA/PBS (300 μl/well) for 2 hrs at room temperature (RT). Blocking solution was discarded and anti-EphB4 Fabs were added to the wells at a concentration of 100 μg/mL in 1% BSA/PBS and incubated for 30 min at RT. Recombinant biotinylated murine EphrinB2 was added at a final concentration of 250 ng/mL and incubated at RT for a further 30 min. The plate was washed and bound EphrinB2 was detected with Streptavidin-HRP (incubation 30 min at RT) and chemifluorescent substrate.

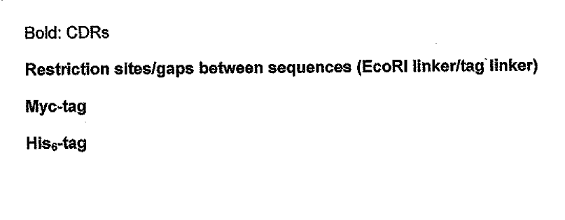

Result: see FIG. 8: EphrinB2 does not bind to EphA4 (low/background signal), but to EphB3 (specific interaction, but not inhibited by anti-EphB4 HuCAL Fabs) and EphB4 (specific interaction, inhibited by HuCAL Fab).

### Example 9

### FACS Binding to hEphB4 Expressing CHO Cells

CHO cells transfected with a myc-tagged hEphB4 receptor (Sturz et al., 2004 Biochem Biophys Res Commun. 313(1):80-8) were trypsinized, washed and resuspended in FACS buffer (PBS/1% BSA) containing anti-EphB4 antibody at a concentration of 10 μg/mL and incubated at 4° C. for 1 hr. Cells were washed in FACS buffer and resuspended in FACS buffer containing goat anti-human IgG (FITC) diluted 1:100 (Jackson Immunoresearch, #109-096-088) and incubated at 4° C. for 1 hr. After a final wash step, cell staining was analyzed in a Becton Dickinson FACS.

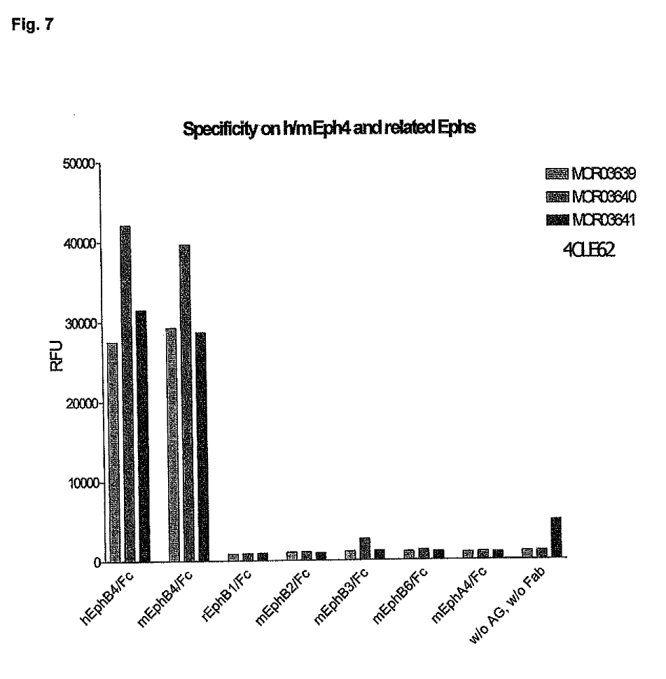

Result: see FIG. 9: anti-EphB4 Fab MOR03640 binds hEphB4-transfected CHO cells

### Example 10

### Inhibition of mEphrinB2-mEphB4 Interaction by Anti-EphB4 Fab

A 96-well plate was coated with recombinant mEphB4 in PBS o/n at 4° C. (5 μg/mL; 100 μL/well). The next day, the antigen was discarded and the plate blocked with 3% BSA/PBS (300 μl/well) for 2 hrs at room temperature (RT). Blocking solution was discarded and anti-EphB4 Fabs were added to the wells at different concentrations in 1% BSA/PBS and incubated for 30 min at RT. Recombinant biotinylated murine EphrinB2 was added at a final concentration of 250 ng/mL and incubated at RT for a further 30 min. The plate was washed and bound EphrinB2 was detected with Streptavidin-HRP (incubation 30 min at RT) and chemifluorescent substrate.

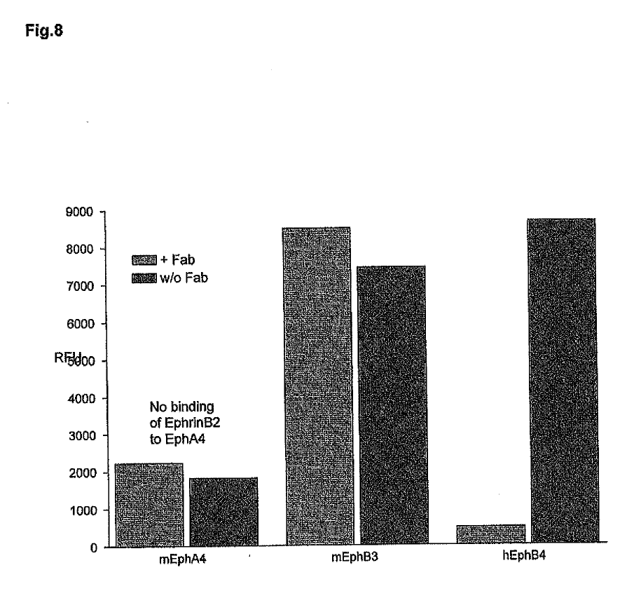

Result: see FIG. 10: Inhibition of mEphrinB2 binding to mEphB4 by anti-EphB4 Fabs.

### Example 11

### Inhibition of mEphrinB2-hEphB4 Interaction by Anti-EphB4 Fab

A 96-well plate was coated with recombinant hEphB4 in PBS o/n at 4° C. (5 μg/mL; 100 μL/well). The next day, the antigen was discarded and the plate blocked with 3% BSA/PBS (300 μl/well) for 2 hrs at room temperature (RT). Blocking solution was discarded and anti-EphB4 Fabs were added to the wells at different concentrations in 1% BSA/PBS and incubated for 30 min at RT. Recombinant biotinylated murine EphrinB2 was added at a final concentration of 250 ng/mL and incubated at RT for a further 30 min. The plate was washed and bound EphrinB2 was detected with Streptavidin-HRP (incubation 30 min at RT) and chemifluorescent substrate.

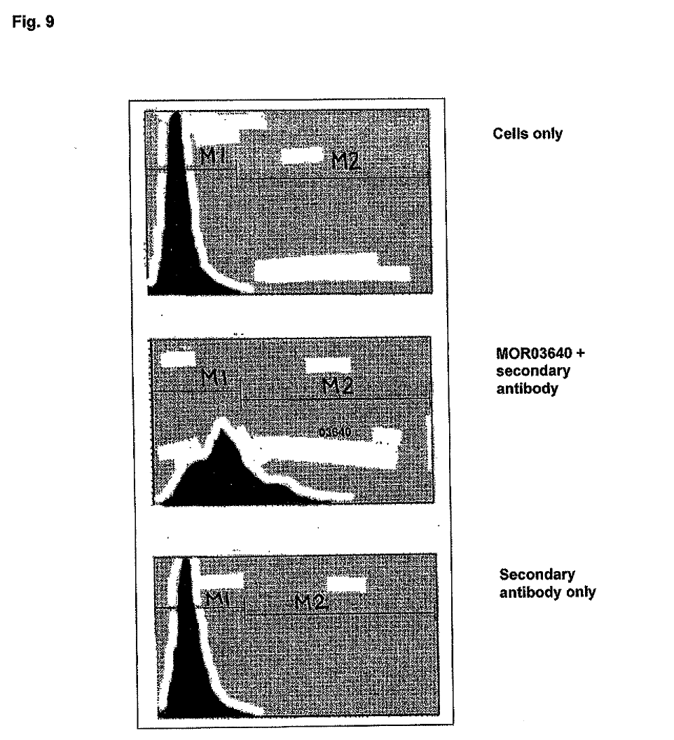

Result: see FIG. 11: Inhibition of mEphrinB2 binding to hEphB4 by anti-EphB4 Fabs.

### Example 12

### Inhibition of mEphrinB2 Binding to CHO-hEphB4 Cells

CHO-hEphB4 cells were trypsinized, washed and resuspended in FACS buffer (PBS/1% BSA) containing anti-EphB4 antibody at different concentrations and incubated at 4° C. for 30 min. Cells were washed in FACS buffer and resuspended in FACS buffer containing biotinylated mEphrinB2 at 250 ng/mL and incubated at 4° C. for 1 hr. cells were washed in FACS buffer and resuspended in FACS buffer containing Streptavidin-PE and incubated at 4° C. for 30 min. After a final wash step, cell staining was analyzed in a Becton Dickinson FACS.

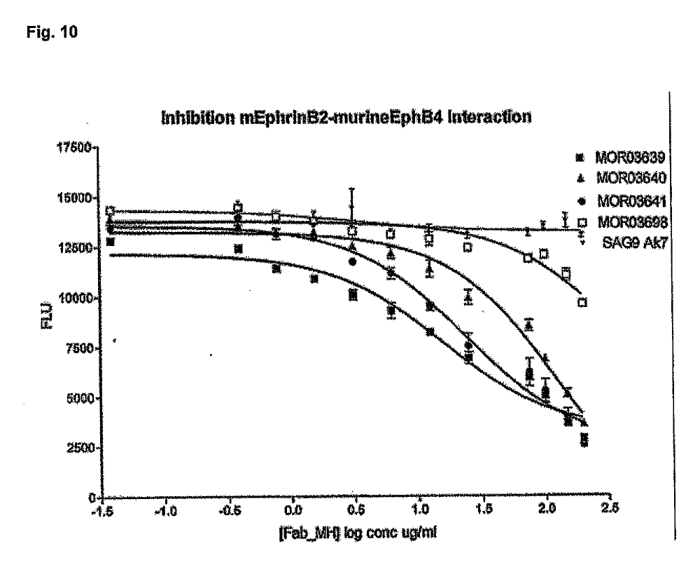

Result: see FIG. 12: with increasing concentration, anti-EphB4 Fabs inhibit EphrinB2 binding to hEphB4-transfected CHO cells.

### Example 13

### Results for Affinity Matured Antibodies

Aim of this approach was to improve the affinity of EphB4 binders. Therefore CDR-optimized sub-libraries were prepared. Also derivatives of MOR03640 were generated (MOR07308, MOR07309, MOR0710, MOR07311). The panning strategy was to pan with those sublibraries under stringent conditions.

### 13.2 Generation of Maturation Libraries

The following five parental Fab fragments were chosen for the generation of maturation libraries:

TABLE 5
Overview of 10 different maturation libraries

| Parental Fabs | VH | VL | LCDR3 | HCDR2 |
|---|---|---|---|---|
| MOR03639 | VH3 | lambda3 | ✓ | ✓ |
| MOR03641 | VH1A | kappa1 | ✓ | ✓ |
| MOR07308 | VH3 | lambda3 | ✓ | ✓ |
| MOR07309 | VH3 | lambda3 | ✓ | ✓ |
| MOR07310 | VH3 | lambda3 | ✓ | ✓ |

The Panning was performed via Fc-capture (human EphB4-Fc (round 1), mouse EphB4-Fc (round 2) and human EphB4-Fc (round 3)), direct (human EphB4 (round 1), mouse EphB4-Fc (round 2, direct) and human EphB4 (round 3), differential whole cell panning (DWCP) (CHO-EphB4 (round 1), mouse EphB4-Fc (round 2, direct) and CHO-EphB4 (round 3)) or via whole cell panning (CHO-EphB4 (round 1), CHO-EphB4 (round 2) and CHO-EphB4 (round 3)). After ARF (affinity ranking factor) screening, SET (solution equilibrium titration), FACS screening on CHO-EphB4 cells and specifity and cross reactivity testing against murine EphB4-Fc 25 Fabs were expressed and purified in an adequate quality. Binders with higher affinity could be isolated, for the corresponding CDRs of MOR07712-MOR07739 see Table 6.

TABLE 6
31 affinity matured EphB4-antibody_CDR-sequences:

| | | | | | | |
|---|---|---|---|---|---|---|
| MOR # | LCDR1 | LCDR2 | LCDR3 | HCDR1 | HCDR2 | HCDR3 |

| | | | | | | | |
|---|---|---|---|---|---|---|---|
| MOR07712 | RASQNINNFLN | LLIYGSSILQS | HQVSDTSD | GGTFSTYAIS | WMGAIIPIWGDAWYAQKFQG | SSYTILTTYYNYNAMDN | |
| MOR07713 | RASQNINNFLN | LLIYGSSILQS | HQVSDTSD | GGTFSTYAIS | WMGDIIPIWGDARYAQKFQG | SSYTILTTYYNYNAMDN | |
| MOR07714 | RASQNINNFLN | LLIYGSSILQS | HOVSDTSD | GGTFSTYAIS | WMGEIIPIFGEADYAQKFQG | SSYTILTTYYNYNAMDN | |
| MOR07715 | RASQNINNFLN | LLIYGSSILQS | HQVSDTSD | GGTFSTVAIS | WMGKIIPIFGDADYAQKFQG | SSYTILTTYYNYNAMDN | |
| MOR07716 | RASQNINNFLN | LLIYGSSILQS | HQVSDTSD | GGTFSTYAIS | WMGOIIPIFGDADYAQKFQG | SSYTILTTYYNYNAMDN | |
| MOR07718 | RASQNINNFLN | LLIYGSSILQS | HQVSDTSD | GGTFSTYAIS | WMGSIIPIFGDADYAQKFQG | SSYTILTTYYNYNAMDN | |
| MOR07719 | SGDNLGKKYVH | LVIYYDSKRPS | SAWTTTMMHI | GFTFNNAWMS | WVSLIEAKEANGATSYAAGVKG | KGMSFYNNKYAKSMFDL | |
| MOR07720 | SGDNLGKKYVH | LVIYYDSKRPS | SAWTTTMMHI | GFTFNNAWMS | WVSQIEAKQPGGATSYAASVKG | KGMSFYNNKYAKSMFDL | |
| MOR07721 | SGDNLGKKYVH | LVIYYDSKRPS | SAWTTTMMHI | GFTFNNAWMS | WVSTIEAKMPNGATFYAAPVKG | KGMSFYNNKYAKSMFDL | |
| MOR07722 | SGDNLGKKYVH | LVIYYDSKRPS | SAWTTTMMHI | GFTFNNAWMS | WVSAINAKGQFTSYADSVKG | KGMSFYNNKYAKSMFDL | |
| MOR07724 | SGDNLGKKYVH | LVIYYDSKRPS | SAWTTTMMHI | GFTFNNAWMS | WVSGISADGVHKFYADSVKG | KGMSFYNNKYAKSMFDL | |
| MOR07725 | SGDNLGKKYVH | LVIYYDSKRPS | SAWTTTMMHI | GFTFNNAWMS | WVSGISADGVHKFYADSVKG | KGMSFVNNKYAKSMFDL | |
| MOR07726 | RASQNINNFLN | LLIYGSSILQS | SQDHFYPPSG | GGTFSTYAIS | WMGRIIPIFGTANYAQKFQG | SSYTILTTYYNYNAMDN | |
| MOR07728 | RASQNINNFLN | LLIYGSSILQS | QQNDFYSPS | GGTFSTYAIS | WMGRIIPIFGTANYAQKFQG | SSYTILTTYYNYNAMDN | |
| MOR07729 | RASQNINNFLN | LLIYGSSILQS | QQYDFYLSG | GGTFSTYAIS | WMGRIIPIFGTANYAQKFQG | SSYTILTTYYNYNAMDN | |
| MOR07730 | RASQNINNFLN | LLIYGSSILQS | IQTFFLPD | GGTFSTYAIS | WMGRIIPIFGTANYAQKFQG | SSYTILTTYYNYNAMDN | |
| MOR07731 | RASQNINNFLN | LLIYGSSILQS | QQQDNLPR | GGTFSTYAIS | WMGRIIPIFGTANYAQKFQG | SSYTILTTYYNYNAMDN | |
| MOR07732 | RASQNINNFLN | LLIYGSSILQS | QQESYVVT | GGTFSTYAIS | WMGRIIPIFGTANYAQKFQG | SSYTILTTYYNYNAMDN | |
| MOR07733 | RASQNINNFLN | LLIYGSSILQS | QQTHSQST | GGTFSTYAIS | WMGRIIPIFGTANYAQKFQG | SSYTILTTYYNYNAMDN | |
| MOR07734 | SGDNLGKKYVH | LVIYYDSKRPS | QAWGAGSHQM | GFTFNNAWMS | WVSNITSSSSMTHYADSVKG | KGMSFYNNKYAKSMFDL | |
| MOR07735 | SGDNLGKKYVH | LVIYYDSKRPS | QAYDVNYMQD | GFTFSNSWMS | WVSNITSSSSMTHYADSVKG | KGMSFVNNKVAKSMFDL | |
| MOR07736 | SGDNLGKKYVH | LVIYYDSKRPS | QTWTLSHMGN | GFTFNNAWMS | WVSNITSSSSMTHYADSVKG | KGMSFYNNKVAKSMFDL | |
| MOR07737 | SGDNLGKKYVH | LVIYYDSKRPS | SSWDSNVVSI | GFTFNNAWMS | WVSNITSSSSMTHYADSVKG | KGMSFYNNKVAKSMFDL | |
| MOR07738 | SGDNLGKKYVH | LVIYYDSKRPS | STYDVGSDYY | GFTFSNSWMS | WVSNITSSSSMTHYADSVKG | KGMSFYNNKYAKSMFDL | |
| MOR07739 | SGDNLGKKYVH | LVIYYDSKRPS | QSYAGSFFD | GFTFNNAWMS | WVSNITSSSSMTHYADSVKG | KGMSFYNNKYAKSMFDL | |
| MOR07953 | SGDNLGKKYVH | LVIYYDSKRPS | QTWTLSHMGN | GFTFNNAWMS | WVSAINAKGQFTSYADSVKG | KGMSFYNNKYAKSMFDL | derived |
| | | | | | | | from |
| | | | | | | | 7722 + |
| | | | | | | | 7738 |
| MOR07954 | SGDNLGKKYVH | LVIYYDSKRPS | QAWGAGSHQM | GFTFNNAWMS | WVSAINAKGQFTSYADSVKG | KGMSFYNNKYAKSMFDL | derived |
| | | | | | | | from |
| | | | | | | | 7722 + |
| | | | | | | | 7734 |
| MOR07955 | RASQNINNFLN | LLIYGSSILQS | SQDHFYPPSG | GGTFSTYAIS | WMGKIIPIFGDADYAQKFQG | SSYTILTTYYNYNAMDN | derived |
| | | | | | | | from |
| | | | | | | | 7715 + |
| | | | | | | | 7726 |
| MOR07956 | RASQNINNFLN | LLIYGSSILQS | QQYDFYLSG | GGTFSTYAIS | WMGKIIPIFGDADYAQKFQG | SSYTILTTYYNYNAMDN | derived |
| | | | | | | | from |
| | | | | | | | 7715 + |
| | | | | | | | 7729 |
| MOR07957 | RASQNINNFLN | LLIYGSSILQS | QQNDFYSPS | GGTFSTYAIS | WMGQIIPIFGDADYAQKFQG | SSYTILTTYYNYNAMDN | derived |
| | | | | | | | from |
| | | | | | | | 77l6 + |
| | | | | | | | 7728 |
| MOR07958 | RASQNINNFLN | LLIYGSSILQS | QQYDFYLSG | GGTFSTYAIS | WMGQIIPIFGDADYAQKFQG | SSYTIITTYYNYNAMDN | derived |
| | | | | | | | from |
| | | | | | | | 7716 + |
| | | | | | | | 7729 |

### 13.3 Specifity/Cross Reactivity Check of 25 Matured Fab Fragments in ELISA

The 25 matured Fab fragments were analyzed according to specificity/cross-reactivity in ELISA on the following Eph-Receptors (see table 7):

TABLE 7

| | Identity to human |
|---|---|
| Eph receptor | EphB4 (amino acid level) |
| mouse EphB4 | 88.7 |
| mouse EphB3 | 43.7 |
| mouse EphB2 | 40.8 |
| rat EphB1 | 40.1 |
| mouse EphB6 | 38.8 |
| mouse EphA4 | 38.3 |

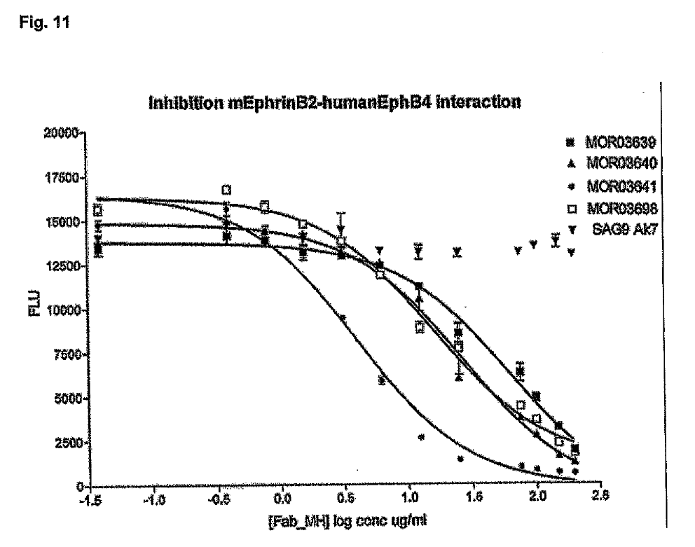

The experiment was repeated twice with comparable results. All matured and parental Fabs bound to human EphB4 at least 10-fold above background, whereas binding to murine EphB4 was different between matured and parental Fabs (see FIG. 13). While signals of parentals MOR03639, 3640, 3641, 7308, 7309 and 7310 on mu EphB4 were only 5-10-fold above background, signals of the matured Fab fragments were at least 10-fold above background (see FIG. 13, part 1 and part 2), suggesting improvement of affinity for matured Fabs. While binding signals of parental Fabs to human EphB4 were already at a saturation level in this assay, binding signals to murine EphB4 lay in a dynamic range, thereby revealing a distinct improvement of the binding capacity of matured Fabs. No significant binding of the matured Fabs to related Eph receptors was detected. The coating control via anti-human IgG Fc-specific antibody revealed that sufficient amounts of all Eph receptors had been immobilized on the ELISA plate.

### 13.4 Pool-Cross-Cloning of HCDR2 and LCDR3 Improved Clones

X-cloning of two Fab fragments derived from the same parental Fab which are either optimized in HCDR2 or in LCDR3 holds potential for further improvement of affinity and/or bioactivity by combining both optimized Fab chains. For this reason cross-cloning was performed of matured Fabs for both pools, MOR03641 and MOR07310. The light chains of HCDR2-matured binders were excised and replaced by the light chains of LCDR3-matured clones from the MOR07310 and MOR03641 groups, respectively. Finally 6 unique x-cloned Fabs were identified and characterized (see Table 6 and 8).

TABLE 8
Unique Fabs after cross-cloning. Unique sequences in the respective matured CDRs
are represented by different coloring

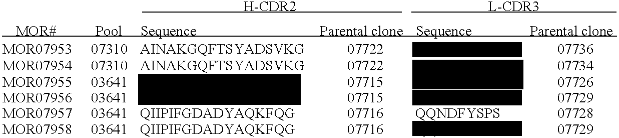

### 13.5 Characterization of Affinity Matured and Cross-Cloned Fab Fragments

### 13.5.1 EC₅₀ Determination by FACS Titration

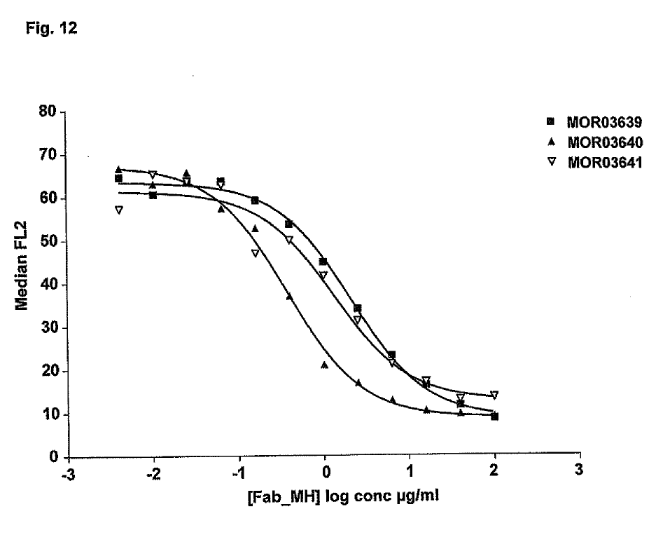

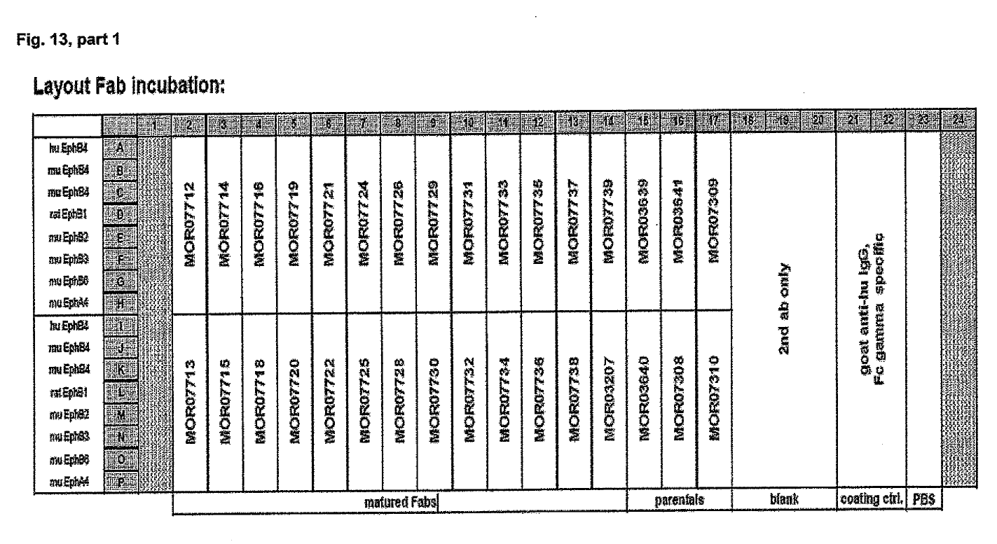

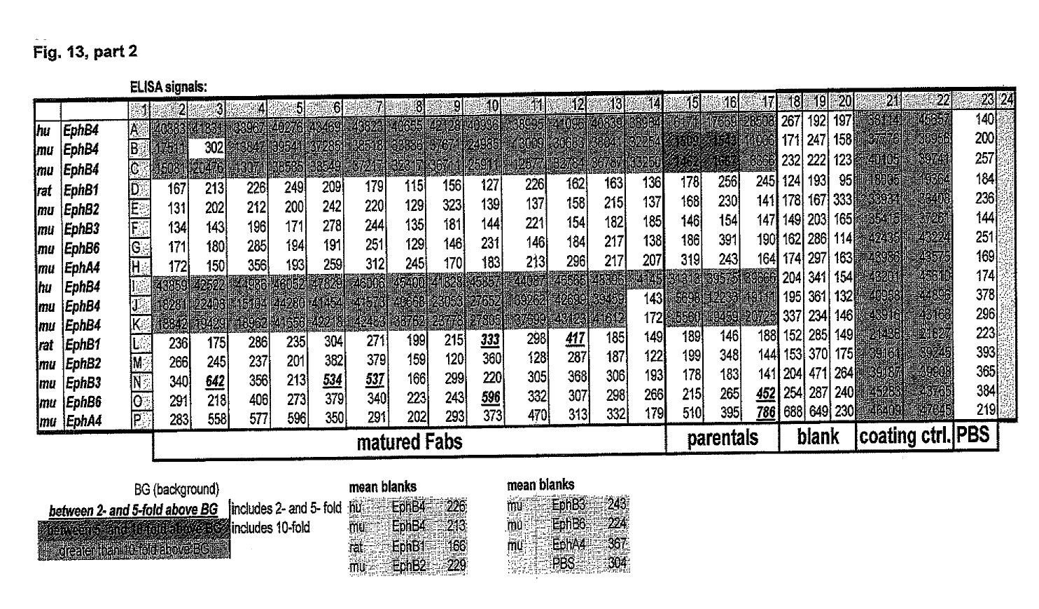

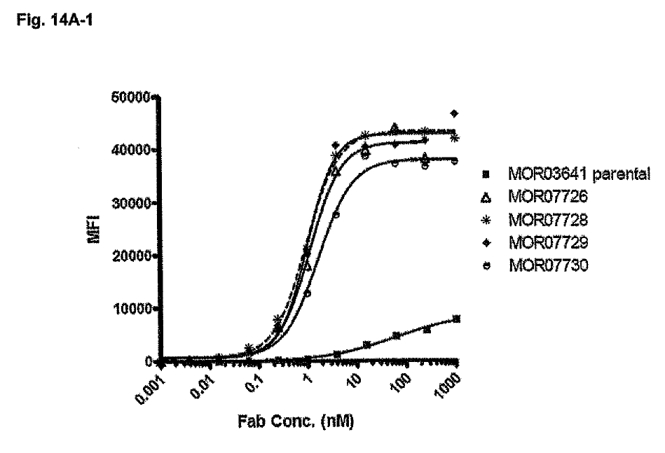

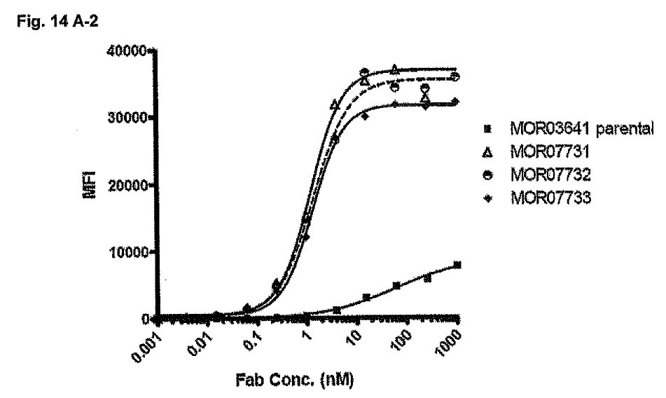

EC₅₀ values were determined in FACS on CHO-EphB4 cells. The whole set of 25 matured binders as well as the 6×-cloned binders were analyzed. For results see FIG. 14 A-1 to B-2 and Table 9.

### 13.5.2 IC₅₀ Determination by FACS Titration

IC50 values (i.e. Fab inhibition of ligand binding) of the 25 matured and the 6×-cloned binders were determined in FACS. CHO-EphB4 cells were preincubated with matured Fab at different concentrations followed by addition of the ligand, murine ephrin-B2 (extracellular domain, 98% identity to human Ephrin-B2). Receptor-bound ephrin-B2 was then detected via biotinylated anti-mouse ephrin-B2 antibody (R&D Systems) and streptavidin-PE. Average IC50 values of at least 2 independent measurements (n>2) are shown in Table 9. All tested Fabs showed inhibitory activity, IC50 values were ranging from 0.3 to 2.9 nM. In both pools (MOR03641 and MOR07310) similar IC50 improvement was observed, i.e. 2-3 fold compared to the corresponding parental. The MOR07310 derivatives perform slightly better than the MOR03641 derivatives. The IC50 values of the x-cloned binders are in the same range as those of their cloning parentals.

TABLE 9
EC₅₀ and IC₅₀ determination of
matured Fabs on CHO-EphB4 cells

| | | | EC50 (nM), | IC50 (nM), |
|---|---|---|---|---|
| | | mat. Fabs | n = 2-4 | n = 2 |
| MOR03641 | H-CDR2 mat. | MOR07712 | 0.79 | 1.36 |
| parental | | MOR07713 | 0.96 | 1.04 |
| | | MOR07714 | 0.86 | 1.88 |
| | | MOR07715 | 1.04 | 1.24 |
| | | MOR07716 | 1.15 | 1.90 |
| | | MOR07718 | 0.92 | 1.30 |
| MOR07310 | H-CDR2 mat. | MOR07719 | 0.60 | 0.94 |
| parental | | MOR07720 | 0.31 | 0.61 |
| | | MOR07721 | 0.48 | 0.97 |
| | | MOR07722 | 0.75 | 1.46 |
| | | MOR07724 | 0.33 | 0.51 |
| | | MOR07725 | 0.38 | 0.78 |
| MOR03641 | L-CDR3 mat. | MOR07726 | 1.20 | 2.87 |
| parental | | MOR07728 | 1.01 | 1.49 |
| | | MOR07729 | 1.39 | 1.46 |
| | | MOR07730 | 1.74 | 1.84 |
| | | MOR07731 | 1.51 | 2.39 |
| | | MOR07732 | 1.28 | 2.29 |
| | | MOR07733 | 1.22 | 1.31 |
| MOR07310 | L-CDR3 mat. | MOR07734 | 0.55 | 0.90 |
| parental | | MOR07735 | 0.28 | 0.34 |
| | | MOR07736 | 0.70 | 0.53 |
| | | MOR07737 | 0.73 | 1.17 |
| | | MOR07738 | 0.44 | 0.62 |
| | | MOR07739 | 0.31 | 0.67 |
| MOR07310 | cross cloned | MOR07953 | 0.77 | 0.87 |
| parental | H-CDR2 and | MOR07954 | 0.84 | 0.85 |
| MOR03641 | L-CDR3 | MOR07955 | 0.98 | 1.25 |
| parental | | MOR07956 | 0.76 | 2.23 |
| | | MOR07957 | 0.60 | 1.7 |
| | | MOR07958 | 0.43 | 1.13 |
| | | MOR03641 | >40 | 4.70 |
| | | MOR07310 | 0.73 | 1.26 |

### 13.5.3 Affinity Determination by Biacore and Set Measurement

Initially Biacore measurement on hu EphB4-Fc was performed for the 25 matured Fab fragments, to check whether this method allows affinity determination of the affinity-matured Fabs. The calculated KD were below 6.6 nM for all Fabs. However, very low koff rates were observed for several Fabs (resulting in KD<<500 pM), which did not allow an accurate KD determination in Biacore, meaning that in several cases the instrument limit was reached.

Therefore KD values of the 25 matured and the 6×-cloned Fabs were determined using SET (MSD instrument) on recombinant human and murine EphB4-Fc in order to obtain more reliable KD values. Two independent measurements were performed on human and murine EphB4-Fc. Data are summarized in Table 10.

TABLE 10
K_{D} determination using SET (in MSD) on
recombinant human and murine EphB4-Fc.

| | | | | Kd (pM) |
|---|---|---|---|---|
| | | | Kd (pM) | murine |
| | | | human EphB4 | EphB4 |
| | | | n = 2-3; | n = 2; |
| | | mat. Fabs | *n = 1 | *n = 1 |
| MOR03641 | H-CDR2 mat. | MOR07712 | 633 | 2738 |
| parental | | MOR07713 | 1035* | 2688* |
| | | MOR07714 | 363 | 1553 |
| | | MOR07715 | 393 | 2256 |
| | | MOR07716 | 924 | 3131 |
| | | MOR07718 | 664 | 3106 |
| MOR07310 | H-CDR2 mat. | MOR07719 | 64 | 127 |
| parental | | MOR07720 | 50 | 49 |
| | | MOR07721 | 114 | 163 |
| | | MOR07722 | 125 | 73 |
| | | MOR07724 | 47 | 138 |
| | | MOR07725 | 71 | 147 |
| MOR03641 | L-CDR3 mat. | MOR07726 | 314 | 851 |
| parental | | MOR07728 | 58 | 104 |
| | | MOR07729 | 87 | 192 |
| | | MOR07730 | 677* | 2214* |
| | | MOR07731 | 351 | 516 |
| | | MOR07732 | 286 | 996 |
| | | MOR07733 | 1043 | 3361 |
| MOR07310 | L-CDR3 mat. | MOR07734 | 63 | 79* |
| parental | | MOR07735 | 27 | 152 |
| | | MOR07736 | 59 | 52* |
| | | MOR07737 | 64 | 216 |
| | | MOR07738 | 31 | 61 |
| | | MOR07739 | 202 | 648 |
| MOR07310 | cross cloned | MOR07953 | 39 | 49 |
| parental | H-CDR2 and | MOR07954 | 18 | 108 |
| MOR03641 | L-CDR3 | MOR07955 | 22 | 130 |
| parental | | MOR07956 | 14 | 58 |
| | | MOR07957 | 30 | 49 |
| | | MOR07958 | 33 | 180 |
| | | MOR03641 | >5000 | n.d. |
| | | MOR07310 | 254 | 1413 |
| | | MOR03640 | 1054 | 6635* |

The measured KD on human EphB4-Fc ranged between 14 pM and ˜1 nM, while the KD on murine EphB4-Fc ranged between 49 pM and ˜3 nM. In addition, the x-cloned binders show an up to 14-fold improved affinity for human EphB4 (e.g. MOR07955) and an up to 6-fold improvement for murine EphB4-Fc (e.g. MOR07956) compared to their cloning-parentals.

### 13.5.4 Inhibition of EphrinB2 Induced Receptor Phosphorylation by Affinity Maturated Fab-Fragments

CHO cells were transfected with human EphB4 containing a myc-tag. Transfected cells were seeded into 96-well microtiter plates and incubated at 37° C., 5% CO₂ for 24 hours. Monomeric EphB4 Fabs were added to the wells in different concentrations and incubated for 30 [p1] minutes, then EphrinB2-Fc was added to the wells in a final concentration of 250 ng/mL[p2]. After a further incubation for 30[p3] minutes, the cells were washed once with PBS and then lysed. EphB4 in the lysates was captured via its myc-tag. Phosphorylation was detected with a peroxidase-coupled anti-phospho-tyrosine antibody.

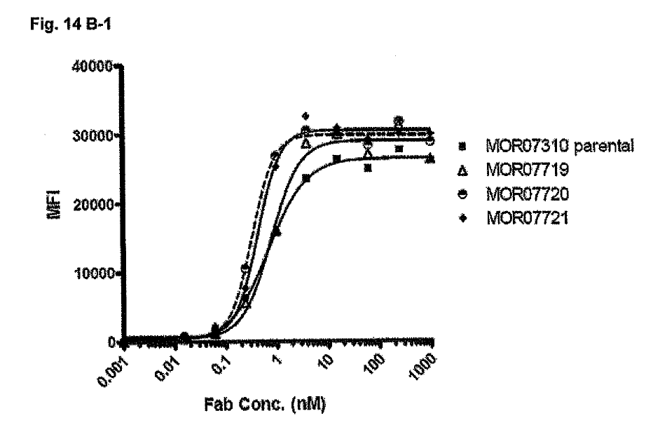

The results are shown in FIG. 15, part 1 and part 2. The ligand-induced phosphorylation (activation) of the EphB4 receptor on the transfected CHO cells is measured via a peroxidase-coupled anti-phospho-tyrosine antibody. Positive control: 100% phosphorylation of the receptor by EphrinB2 alone, negative control: EphrinB2 plus an irrelevant Fab MOR03268 antibody; this Fab does not bind to the EphB4 receptor, thus does not inhibit binding of the EphrinB2 ligand to the receptor and therefore also leads to 100% phosphorylation of the receptor. Anti-EphB4 Fabs inhibit intracellular EphB4 phosphorylation with IC50 between 0.85 and 2.1 μg/mL (17 and 42 nM)

13.6 Conversion of 12 Optimized Fab Fragments into the IgG4 Pro-Format and Characterization
13.6.1 Conversion of Optimized Fab Fragments into the IgG4 Pro-Format

12 affinity matured Fabs, including all 6×-cloned binders, were selected for conversion into human IgG4 Pro-formate, mainly based on the affinity data. In addition following criteria were taken into account: The sequence diversity in CDRs and clones from both HCDR2 and LCDR3 maturation were selected. Following clones were selected for conversion: MOR07720, 7721, 7724, 7725, 7728, 7729, 7953 (7722×7736), 7954 (7722×7734), 7955 (7715×7726), 7956 (7715×7729), 7957 (7716×7728), 7958 (7716×7729).

### 13.6.1 EC₅₀ Determination by FACS Titration

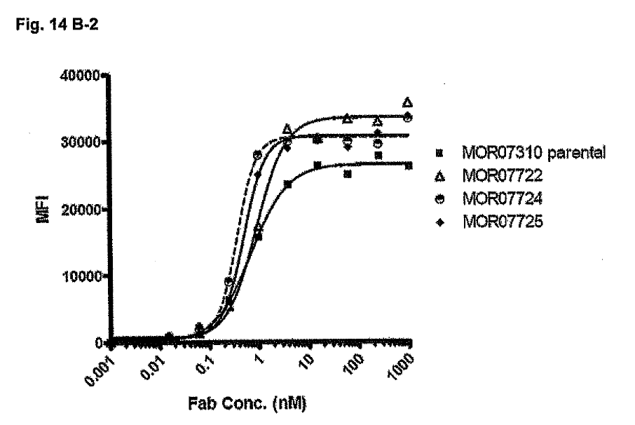

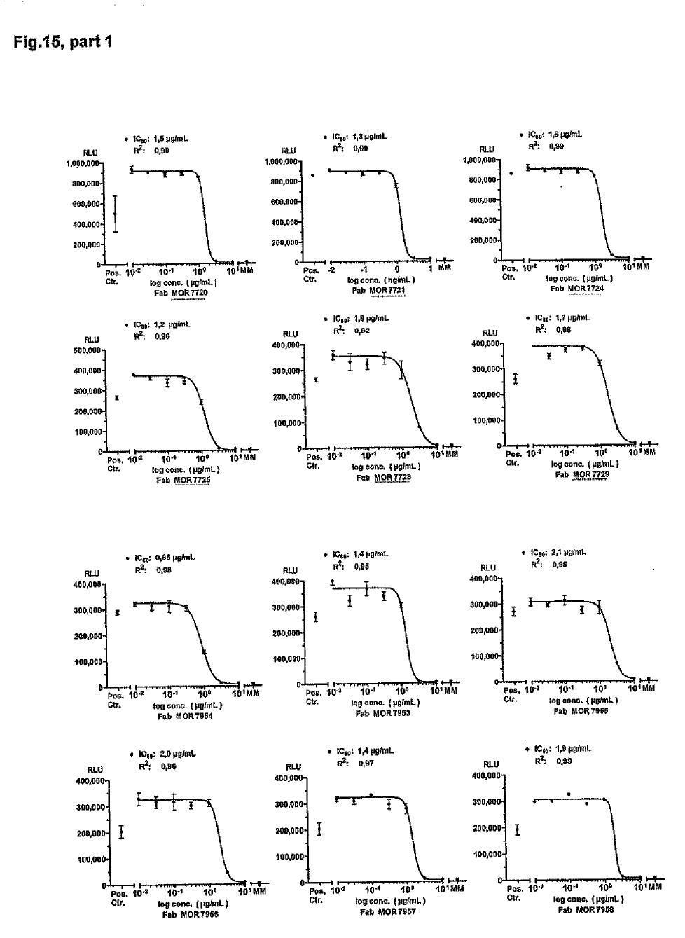

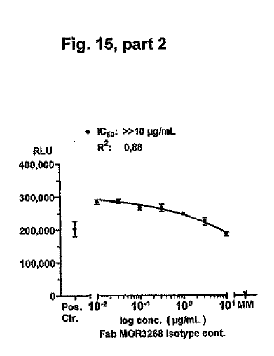

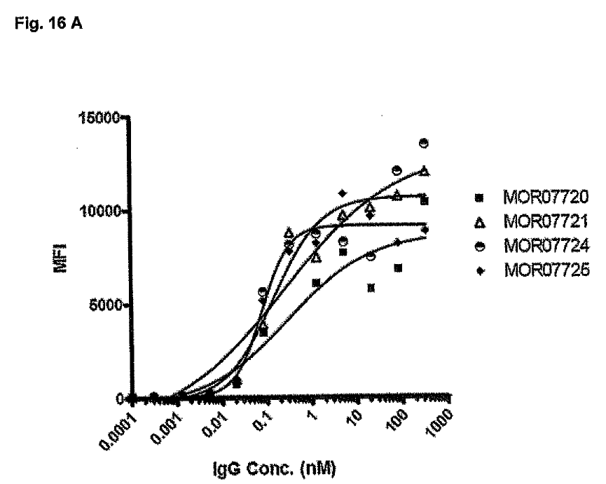

For the determination of EC50 values, the 12 final IgGs were analyzed on CHO-EphB4 cells in FACS. The EC50 data of one representative experiment are presented in FIG. 16A to C. The EC50 values of the IgGs, determined in two independent experiments, are given in Table 11 and are compared to the EC50 values of the corresponding Fab. The EC50 values of the IgGs are slightly better than those of the Fabs, with 3 exceptions, and are ranging from 0.08 nM to 4.8 nM. Again, as already seen for the Fab, the EC50 values of the MOR07310 pool were slightly better than those of the MOR03641 pool.

### 13.6.2 IC₅₀ Determination by FACS Titration

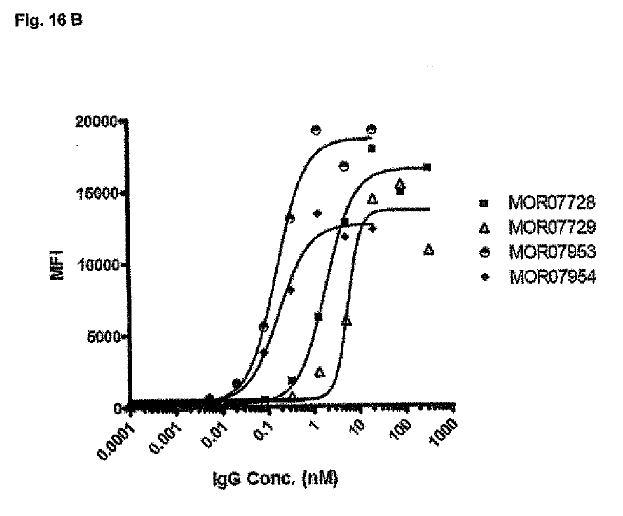

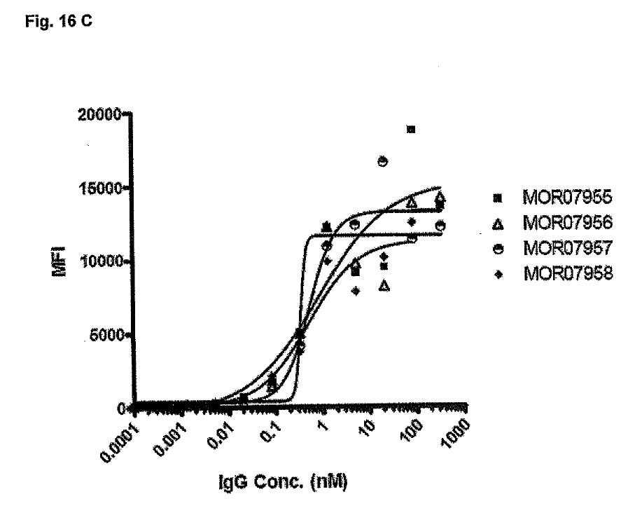

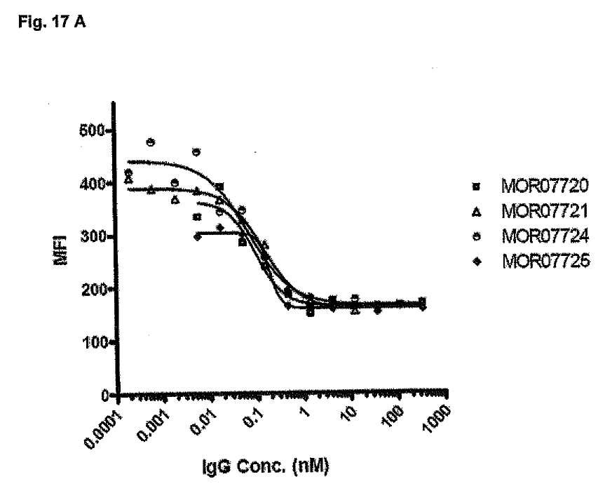

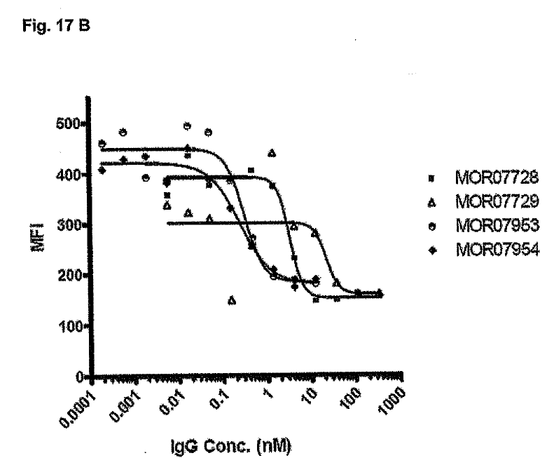

C50 values (i.e. IgG4-Pro inhibition of ligand binding) were determined in FACS. 12×104 CHO-EphB4 cells were preincubated with IgGs (12 final candidates) at different concentrations followed by addition of the ligand, murine ephrin-B2. Receptor-bound ephrin-B2 was detected via biotinylated anti-mouse ephrin-B2 antibody (R&D Systems) and streptavidin-PE. The IC50 data of one representative measurement are presented in FIGS. 17A to C. The IC50 values of the IgGs, determined in two independent experiments, are given in Table 11 and are compared to the IC50 values of the corresponding Fab. Except for the binder MOR07728 and -29, the IC50 values of the IgGs were up to 6-fold improvement compared to the corresponding Fab (e.g. MOR07721) and are ranging from 0.10 nM to 11.6 nM. Again, as already seen for the Fab, the IC50 values of the MOR07310 pool were slightly better than those of the MOR03641 pool.

TABLE 11
EC₅₀ and IC₅₀ determination of 12 affinity-matured IgG4-Pro's on CHO-EphB4 cells

| | | | maturated IgG4-Pro | | | |
|---|---|---|---|---|---|---|
| | | | EC50 (nM), n = 2, | IC50 (nM), n = 2-3; | maturated Fabs | |
| | | mat. Fabs | *n = 1 | *n = 1 | EC50 (nM), n = 2-4 | 50 (nM), n = 2 |
| MOR03641 | L-CDR3 mat. | MOR07728 | 1.58 | 3.26* | 1.01 | 1.49 |
| parental | | MOR07729 | 4.83 | 11.55 | 1.39 | 1.46 |
| | crosds cloned | MOR07955 | 0.51 | 0.31 | 0.98 | 1.25 |
| | H-CDR2 and | MOR07956 | 0.34 | 0.43* | 0.76 | 2.23 |
| | L-CDR3 | MOR07597 | 0.36 | 0.79 | 0.60 | 1.70 |
| | | MOR07958 | 0.43 | 0.34 | 0.43 | 1.13 |
| MOR07310 | H-CDR2 mat. | MOR07720 | 0.30* | 0.11 | 0.31 | 0.61 |
| parental | | MOR07721 | 0.12 | 0.16 | 0.48 | 0.97 |
| | | MOR07724 | 0.18 | 0.10 | 0.33 | 0.51 |
| | | MOR07725 | 0.08 | 0.16 | 0.38 | 0.78 |
| | cross cloned | MOR07953 | 0.17 | 0.23 | 0.77 | 0.87 |
| | H-CDR2 and | MOR07954 | 0.22 | 0.26 | 0.84 | 0.85 |
| | L-CDR3 | | | | | |

### 13.6.3 Inhibition of EphrinB2 Induced Receptor Phosphorylation by IgG4-Pro Constructs

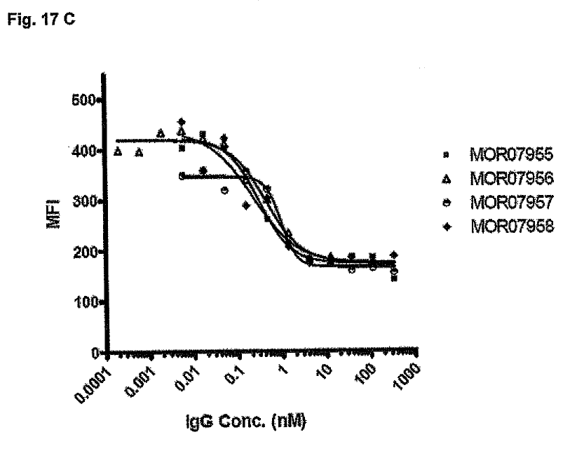

The assay was performed as described under 13.5.4. Typical results are shown in FIG. 18, part 1 and part 2.

All IgG4pro EphB4 antibodies except for MOR07729 result in phosphorylation of EphB4 without EphrinB2 stimulus at a concentration of 10 μg/mL.

13.6.4 Inhibition of cell adhesion in IgG4-Pro format

IgG4-Pros were tested in adhesion assay on different cell lines (HUVECs, HDMVECs, U-2 OS with and without EphB4, PC3-MM2 and MDA-MB-435). In 96 well plates 50 μl per well of double-concentrated antibody are submitted. Per well 10 000 cells in 50 μl cell culture media (DMEM/HAM's F12+5% FCS) are added. All plates are incubated for 4 hours. Aferwards media with unadhered cells is sucked off and wells are washed. Adhered cells are fixed with glutaraldehyde and stained with crystal violet. The amount of bound dye is proportional to the amount of adhered cells. The absorption is measured ad 595 nm. All IgG4-Pros were tested in a concentration of 50 μg/mL (=0.3 μM).

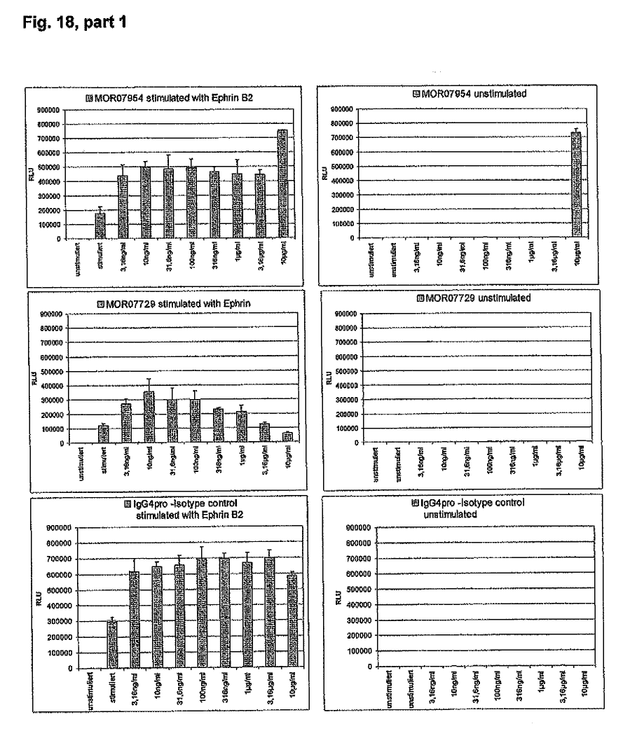

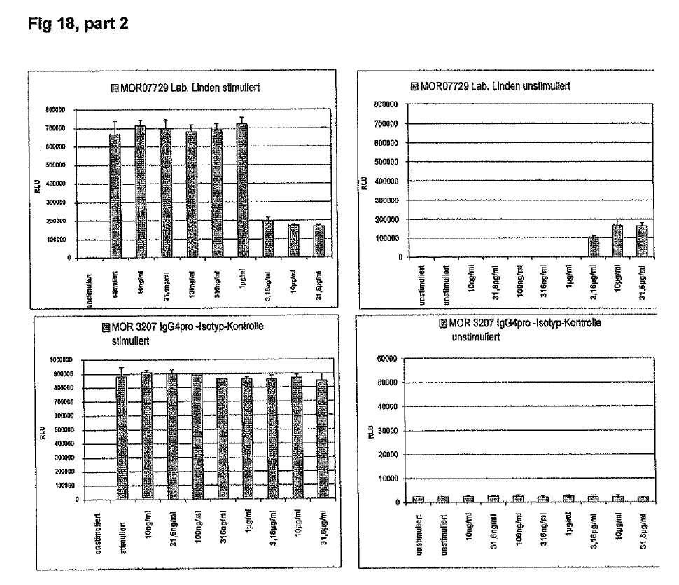

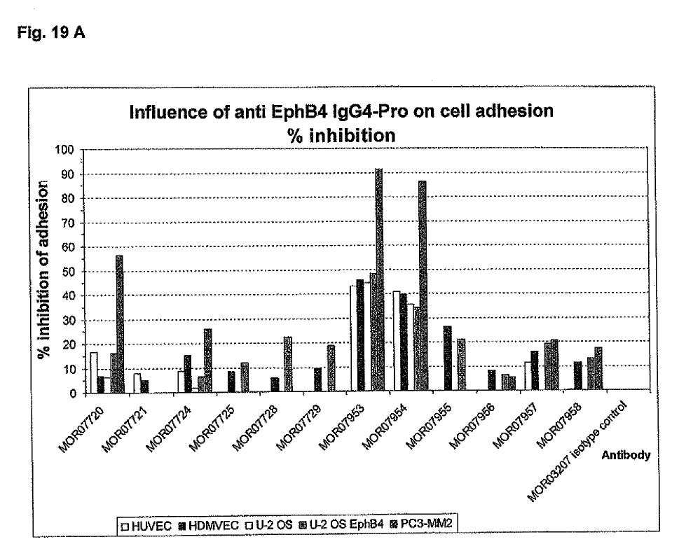

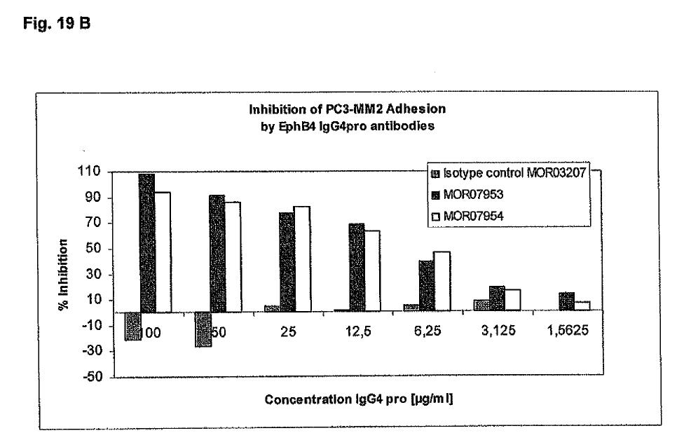

For results see FIGS. 19 A to C.

### 13.6.5 Inhibition of Cell Migration in IgG4-Pro and Fab Format

The assay was designed as a horizontal migration assay. Human cells are plated and grown in a six well plate for 48 h, prior to use the wells are coated with desired protein (Ephrin B2—not in all cases). The confluent monolayer is scraped off in one half of each well (wounding). Then the medium is replaced by starvation medium/0.2% BSA

The test compound (IgG4-Pro or Fab) is added in concentrations as wished. The migration distance of cells into the cell-free part of the well is determined microscopically after a 24 h and a 48 h incubation period. Each data point represents a mean of four regions. PDGF and/or bFGF as a potent chemotactic factor for desired cells is added as positive control. Results: No inhibition of migration was observed using IgG4 07729, weak inhibition of migration was observed using IgG4 07956 (good inhibition for PC3 cells on coated plates). Inhibition of migration was observed using IgG4 07954 (strong for PC3 cells). Coating with ephrin B2 improves inhibitory potential of the antibodies.

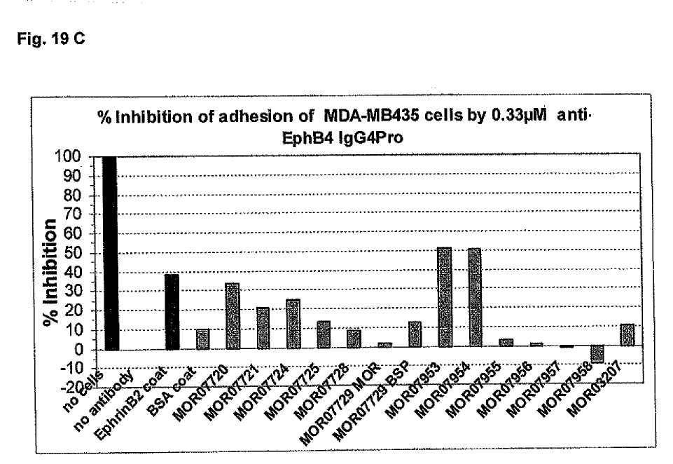

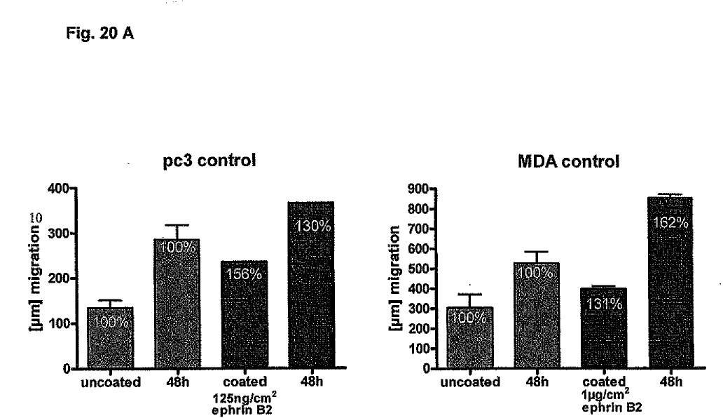

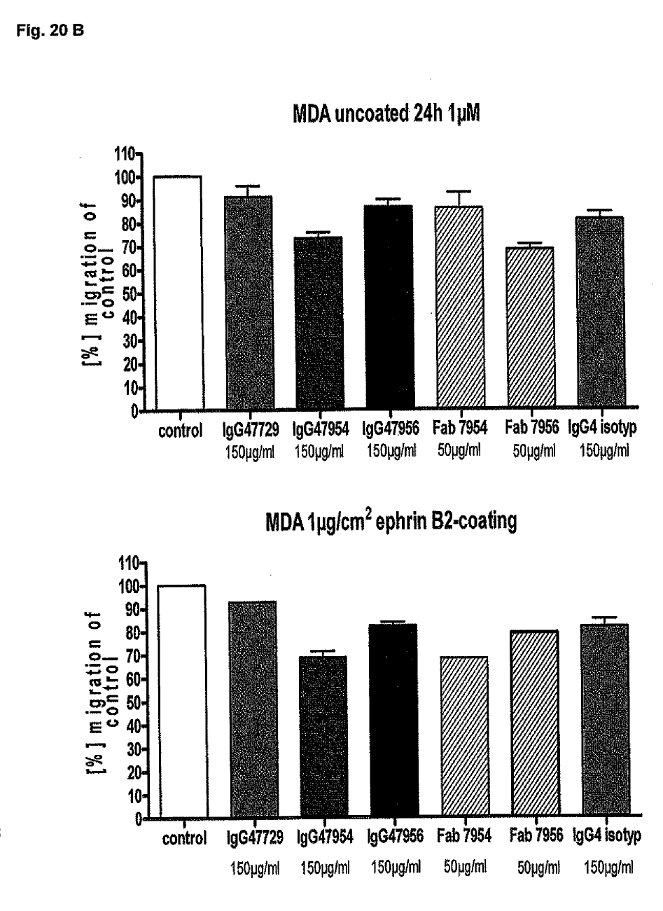

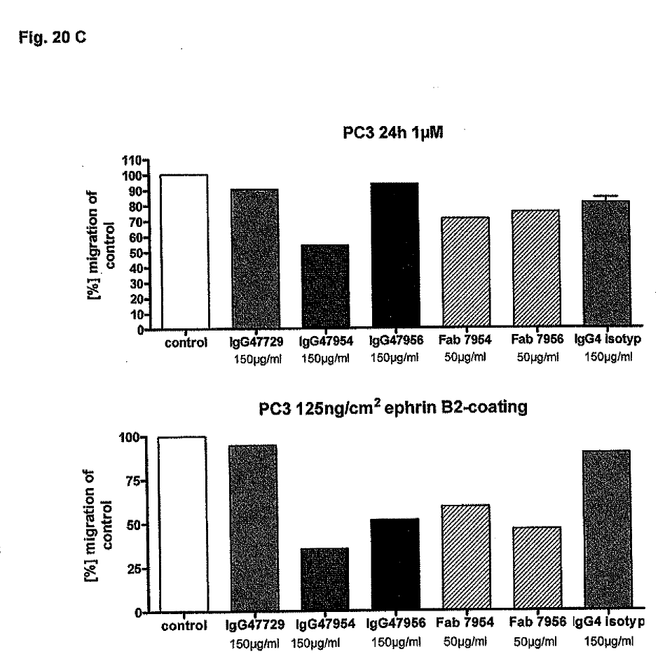

The results are shown in FIGS. 20 A to C.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding European application No. 07075657.2, filed Jul. 31, 2007, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. An antibody or antibody fragment specifically recognizing the EphB4 receptor comprising one or more heavy and light chain sequences selected from the sequences of Seq. ID NO. 1 to Seq. ID NO. 118, or modifications thereof.

2. An antibody or antibody fragment according to claim 1, comprising a complementary determining region (CDR) of a heavy chain (CDR-H) and a light chain (CDR-L), characterized in that the heavy chain (CDR-H) is selected from the group of Seq. ID NO. 1 to 3, Seq. ID NO. 7 to 9 and Seq. ID NO. 13 to 15; and the light chain (CDR-L) is selected from the group of Seq. ID NO. 4 to 6, Seq. ID NO. 10 to 12, and Seq. ID NO. 16 to 18.

3. An antibody or antibody fragment according to claim 2, characterised in that the heavy chain (CDR-H) is selected
as CDR-H1 from the group of Seq. ID NO. 1, Seq. ID NO. 7 and Seq. ID NO. 13, or
as CDR-H2 from the group of Seq. ID NO. 2, Seq. ID NO. 8 and Seq. ID NO. 14, or
as CDR-H3 from the group of Seq. ID NO. 3, Seq. ID NO. 9 and Seq. ID NO. 15.

4. An antibody or antibody fragment according to claim 2, characterised in that the light chain (CDR-L) is selected
as CDR-L1 from the group of Seq. ID NO. 4, Seq. ID NO. 10 and Seq. ID NO. 16, or
as CDR-L2 from the group of Seq. ID NO. 5, Seq. ID NO. 11 and Seq. ID NO. 17, or
as CDR-L3 from the group of Seq. ID NO. 6, Seq. ID NO. 12 and Seq. ID NO. 18.

5. An antibody or antibody fragment according to claim 3, wherein the light chains (CDR-L) is selected
as CDR-L1 from the group of Seq. ID NO. 4, Seq. ID NO. 10 and Seq. ID NO. 16, or
as CDR-L2 from the group of Seq. ID NO. 5, Seq. ID NO. 11 and Seq. ID NO. 17, or
as CDR-L3 from the group of Seq. ID NO. 6, Seq. ID NO. 12 and Seq. ID NO. 18.

6. An antibody or antibody fragment according to claim 1, comprising the variable domain (V) of the complementary determining region (CDR) of a heavy chain (CDR-H, VH) and a light chain (CDR-L, VL), characterized in that the heavy chain (CDR-H) is selected from the group of Seq. ID NO. 19 and the light chain (CDR-L) is selected from the group of Seq. ID NO. 20, or the heavy chain (CDR-H) is selected from the group of Seq. ID NO. 21 and the light chain (CDR-L) is selected from the group of Seq. ID NO. 22, or the heavy chain (CDR-H) is selected from the group of Seq. ID NO. 23 and the light chain (CDR-L) is selected from the group of Seq. ID NO. 24.

7. An antibody or antibody fragment according to claim 1, comprising the variable domain (V) of the complementary determining region (CDR) of a heavy chain (CDR-H, VH) and a light chain (CDR-L, VL), characterized in that the heavy chain (CDR-H) is selected from the group of Seq. ID NO. 19, Seq. ID NO. 21 and Seq. ID NO. 23; and the light chain (CDR-L) is selected from the group of Seq. ID NO. 20, Seq. ID NO. 22 and Seq. ID NO. 24.

8. An antibody or antibody fragment according to claim 1, comprising the constant domain (C) of the complementary determining region (CDR) of a heavy chain (CDR-H, CH) and a light chain (CDR-L, CL), characterized in that the heavy chain (CDR-H) is selected from the group of Seq. ID NO. 25 and the light chain (CDR-L) is selected from the group of Seq. ID NO. 26, or the heavy chain (CDR-H) is selected from the group of Seq. ID NO. 27 and the light chain (CDR-L) is selected from the group of Seq. ID NO. 28, or the heavy chain (CDR-H) is selected from the group of Seq. ID NO. 29 and the light chain (CDR-L) is selected from the group of Seq. ID NO. 30.

9. An antibody or antibody fragment according to claim 1, comprising the constant domain (C) of the complementary determining region (CDR) of a heavy chain (CDR-H, CH) and a light chain (CDR-L, CL), characterized in that the heavy chain (CDR-H) is selected from the group of Seq. ID NO. 25, Seq. ID NO. 27 and Seq. ID NO. 29; and the light chain (CDR-L) is selected from the group of Seq. ID NO. 26, Seq. ID NO. 28 and Seq. ID NO. 30.

10. An antibody or antibody fragment according to claim 1, comprising the complementary determining region (CDR) of a heavy chain (CDR-H) and a light chain (CDR-L) on the Fab fragment, characterized in that the heavy chain (CDR-H, FabH) is selected from the group of Seq. ID NO. 32, Seq. ID NO. 34, Seq. ID NO. 36, Seq. ID NO. 38, Seq. ID NO. 40, Seq. ID NO. 42, Seq. ID NO. 44, Seq. ID NO. 46, Seq. ID NO. 48, Seq. ID NO. 50, Seq. ID NO. 52, Seq. ID NO. 54, Seq. ID NO. 56, Seq. ID NO. 58, Seq. ID NO. 60, Seq. ID NO. 62, Seq. ID NO. 64, Seq. ID NO. 66, Seq. ID NO. 68, Seq. ID NO. 70, Seq. ID NO. 72, Seq. ID NO. 74, Seq. ID NO. 76, Seq. ID NO. 78, Seq. ID NO. 80, Seq. ID NO. 82, Seq. ID NO. 84, Seq. ID NO. 86, Seq. ID NO. 88, Seq. ID NO. 90 and Seq. ID NO. 92; and the light chain (CDR-L, FabL) is selected from the group of Seq. ID NO. 31, Seq. ID NO. 33, Seq. ID NO. 35, Seq. ID NO. 37, Seq. ID NO. 39, Seq. ID NO. 41, Seq. ID NO. 43, Seq. ID NO. 45, Seq. ID NO. 47, Seq. ID NO. 49, Seq. ID NO. 51, Seq. ID NO. 53, Seq. ID NO. 55, Seq. ID NO. 57, Seq. ID NO. 59, Seq. ID NO. 61, Seq. ID NO. 63, Seq. ID NO. 65, Seq. ID NO. 67, Seq. ID NO. 69, Seq. ID NO. 71, Seq. ID NO. 73, Seq. ID NO. 75, Seq. ID NO. 77, Seq. ID NO. 79, Seq. ID NO. 81, Seq. ID NO. 83, Seq. ID NO. 85, Seq. ID NO. 87, Seq. ID NO. 89 and Seq. ID NO. 91.

11. An antibody according to claim 1, characterized in that the heavy chain (IgG4 H) is selected from the group of Seq. ID NO. 94, Seq. ID NO. 96, Seq. ID NO. 98, Seq. ID NO. 100, Seq. ID NO. 102, Seq. ID NO. 104, Seq. ID NO. 106, Seq. ID NO. 108, Seq. ID NO. 110, Seq. ID NO. 112, Seq. ID NO. 114 and Seq. ID NO. 116; and the light chain (IgG4 L) is selected from the group of Seq. ID NO. 93, Seq. ID NO. 95, Seq. ID NO. 97, Seq. ID NO. 99, Seq. ID NO. 101, Seq. ID NO. 103, Seq. ID NO. 105, Seq. ID NO. 107, Seq. ID NO. 109, Seq. ID NO. 111, Seq. ID NO. 113 and Seq. ID NO. 115.

12. An antibody according to claim 1, characterized in that the heavy chain (IgG1 H) is selected from the group of Seq. ID NO. 118 and the light chain (IgG1 L) is selected from the group of Seq. ID NO. 117.

13. An antibody or antibody fragment according to claim 1 wherein the heavy chain is at the C-terminus and the light chain is at the N-terminus or the heavy chain is at the N-terminus and the light chain is at the C-terminus.

14. An antibody or antibody fragment which exhibits a substantially equivalent antigen binding to the EphB4 receptor or which binds to the same epitope on the EphB4 receptor, comparable to an antibody or antibody fragment according to claim 1.

15. The antibody of claim 1, which is monovalent.

16. The antibody of claim 1, which is polyvalent.

17. An antibody or antibody fragment according to claim 1 as a medicament.

18. An antibody or antibody fragment according to claim 1 wherein the antibody or antibody fragment is used in combination with a cancer active compound.

19. A nucleic acid encoding an antibody or antibody fragment of claim 1.

20. The nucleic acid of claim 19, which is a DNA.

21. The nucleic acid of claim 19, which is located on an expression vector.

22. A host cell transformed or transfected with a nucleic acid of claim 19.

23. A method for producing an antibody or antibody fragment of claim 1, comprising the steps:
culturing the host cell in a suitable medium under conditions wherein the antibody encoding nucleic acid is expressed, and recovering the antibody from the host cell or the medium.

24. A method for treating an angiogenically disease comprising administering to a patient an effective amount of an antibody or antibody fragment according to claim 1.

25. A method according to claim 24, characterised in that the disease is cancer.


SEQUENCE LISTING (151 sequences)

SEQ ID NO: 1 - protein, 10 aa, Artificial Sequence
  Description of Artificial Sequence Synthetic peptide
GFTFNNSWMS

SEQ ID NO: 2 - protein, 20 aa, Artificial Sequence
  Description of Artificial Sequence Synthetic peptide
WVSNITSSSSMTHYADSVKG

SEQ ID NO: 3 - protein, 17 aa, Artificial Sequence
  Description of Artificial Sequence Synthetic peptide
KGMSFYNNKYAKSMFDL

SEQ ID NO: 4 - protein, 11 aa, Artificial Sequence
  Description of Artificial Sequence Synthetic peptide
SGDNLGKKYVH

SEQ ID NO: 5 - protein, 11 aa, Artificial Sequence
  Description of Artificial Sequence Synthetic peptide
LVIYYDSKRPS

SEQ ID NO: 6 - protein, 10 aa, Artificial Sequence
  Description of Artificial Sequence Synthetic peptide
SAWTTTMMHI

SEQ ID NO: 7 - protein, 10 aa, Artificial Sequence
  Description of Artificial Sequence Synthetic peptide
GFTFSNYYLH

SEQ ID NO: 8 - protein, 16 aa, Artificial Sequence
  Description of Artificial Sequence Synthetic peptide
GISYSGSSTYYADSVK

SEQ ID NO: 9 - protein, 13 aa, Artificial Sequence
  Description of Artificial Sequence Synthetic peptide
FYQRRQIPHGYDV

SEQ ID NO: 10 - protein, 11 aa, Artificial Sequence
  Description of Artificial Sequence Synthetic peptide
SGDNIGDYYVH

SEQ ID NO: 11 - protein, 11 aa, Artificial Sequence
  Description of Artificial Sequence Synthetic peptide
LVIYRDSNRPS

SEQ ID NO: 12 - protein, 10 aa, Artificial Sequence
  Description of Artificial Sequence Synthetic peptide
GSYDMPFGGT

SEQ ID NO: 13 - protein, 10 aa, Artificial Sequence
  Description of Artificial Sequence Synthetic peptide
GGTFSTYAIS

SEQ ID NO: 14 - protein, 20 aa, Artificial Sequence
  Description of Artificial Sequence Synthetic peptide
WMGRIIPIFGTANYAQKFQG

SEQ ID NO: 15 - protein, 17 aa, Artificial Sequence
  Description of Artificial Sequence Synthetic peptide
SSYTILTTYYNYNAMDN

SEQ ID NO: 16 - protein, 11 aa, Artificial Sequence
  Description of Artificial Sequence Synthetic peptide
RASQNINNFLN

SEQ ID NO: 17 - protein, 11 aa, Artificial Sequence
  Description of Artificial Sequence Synthetic peptide
LLIYGSSILQS

SEQ ID NO: 18 - protein, 8 aa, Artificial Sequence
  Description of Artificial Sequence Synthetic peptide
HQVSDTSD

SEQ ID NO: 19 - protein, 126 aa, Artificial Sequence
  Description of Artificial Sequence Synthetic polypeptide
QVQLVESGGGLVQPGGSLRLSCAASGFTFNNSWMSWVRQAPGKGLEWVSNITSSSSMTHYADSVKGRFTISRDNSKNTLYLQMNSLRAEDTAVYYCARKGMSFYNNKYAKSMFDLWGQGTLVTVSS

SEQ ID NO: 20 - protein, 110 aa, Artificial Sequence
  Description of Artificial Sequence Synthetic polypeptide
DIELTQPPSVSVAPGQTARISCSGDNLGKKYVHWYQQKPGQAPVLVIYYDSKRPSGIPERFSGSNSGNTATLTISGTQAEDEADYYCSAWTTTMMHIVFGGGTKLTVLGQ

SEQ ID NO: 21 - protein, 122 aa, Artificial Sequence
  Description of Artificial Sequence Synthetic polypeptide
QVQLVESGGGLVQPGGSLRLSCAASGFTFSNYYLHWVRQAPGKGLEWVSGISYSGSSTYYADSVKGRFTISRDNSKNTLYLQMNSLRAEDTAVYYCARFYQRRQIPHGYDVWGQGTLVTVSS

SEQ ID NO: 22 - protein, 110 aa, Artificial Sequence
  Description of Artificial Sequence Synthetic polypeptide
DIELTQPPSVSVAPGQTARISCSGDNIGDYYVHWYQQKPGQAPVLVIYRDSNRPSGIPERFSGSNSGNTATLTISGTQAEDEADYYCGSYDMPFGGTVFGGGTKLTVLGQ

SEQ ID NO: 23 - protein, 126 aa, Artificial Sequence
  Description of Artificial Sequence Synthetic polypeptide
QVQLVQSGAEVKKPGSSVKVSCKASGGTFSTYAISWVRQAPGQGLEWMGRIIPIFGTANYAQKFQGRVTITADESTSTAYMELSSLRSEDTAVYYCARSSYTILTTYYNYNAMDNWGQGTLVTVSS

SEQ ID NO: 24 - protein, 109 aa, Artificial Sequence
  Description of Artificial Sequence Synthetic polypeptide
DIQMTQSPSSLSASVGDRVTITCRASQNINNFLNWYQQKPGKAPKLLIYGSSILQSGVPSRFSGSGSGTDFTLTISSLQPEDFAVYYCHQVSDTSDTFGQGTKVEIKRT

SEQ ID NO: 25 - protein, 228 aa, Artificial Sequence
  Description of Artificial Sequence Synthetic polypeptide
QVQLVESGGGLVQPGGSLRLSCAASGFTFNNSWMSWVRQAPGKGLEWVSNITSSSSMTHYADSVKGRFTISRDNSKNTLYLQMNSLRAEDTAVYYCARKGMSFYNNKYAKSMFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKS

SEQ ID NO: 26 - protein, 213 aa, Artificial Sequence
  Description of Artificial Sequence Synthetic polypeptide
DIELTQPPSVSVAPGQTARISCSGDNLGKKYVHWYQQKPGQAPVLVIYYDSKRPSGIPERFSGSNSGNTATLTISGTQAEDEADYYCSAWTTTMMHIVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEA

SEQ ID NO: 27 - protein, 224 aa, Artificial Sequence
  Description of Artificial Sequence Synthetic polypeptide
QVQLVESGGGLVQPGGSLRLSCAASGFTFSNYYLHWVRQAPGKGLEWVSGISYSGSSTYYADSVKGRFTISRDNSKNTLYLQMNSLRAEDTAVYYCARFYQRRQIPHGYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKS

SEQ ID NO: 28 - protein, 213 aa, Artificial Sequence
  Description of Artificial Sequence Synthetic polypeptide
DIELTQPPSVSVAPGQTARISCSGDNIGDYYVHWYQQKPGQAPVLVIYRDSNRPSGIPERFSGSNSGNTATLTISGTQAEDEADYYCGSYDMPFGGTVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEA

SEQ ID NO: 29 - protein, 228 aa, Artificial Sequence
  Description of Artificial Sequence Synthetic polypeptide
QVQLVQSGAEVKKPGSSVKVSCKASGGTFSTYAISWVRQAPGQGLEWMGRIIPIFGTANYAQKFQGRVTITADESTSTAYMELSSLRSEDTAVYYCARSSYTILTTYYNYNAMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKS

SEQ ID NO: 30 - protein, 214 aa, Artificial Sequence
  Description of Artificial Sequence Synthetic polypeptide
DIQMTQSPSSLSASVGDRVTITCRASQNINNFLNWYQQKPGKAPKLLIYGSSILQSGVPSRFSGSGSGTDFTLTISSLQPEDFAVYYCHQVSDTSDTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 31 - protein, 214 aa, Artificial Sequence; identical to SEQ ID NO: 30
  Description of Artificial Sequence Synthetic polypeptide

SEQ ID NO: 32 - protein, 247 aa, Artificial Sequence
  Description of Artificial Sequence Synthetic polypeptide
QVQLVQSGAEVKKPGSSVKVSCKASGGTFSTYAISWVRQAPGQGLEWMGAIIPIWGDAWYAQKFQGRVTITADESTSTAYMELSSLRSEDTAVYYCARSSYTILTTYYNYNAMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFDYKDDDDKGAPHHHHHH

SEQ ID NO: 33 - protein, 214 aa, Artificial Sequence; identical to SEQ ID NO: 30
  Description of Artificial Sequence Synthetic polypeptide

SEQ ID NO: 34 - protein, 247 aa, Artificial Sequence
  Description of Artificial Sequence Synthetic polypeptide
QVQLVQSGAEVKKPGSSVKVSCKASGGTFSTYAISWVRQAPGQGLEWMGDIIPIWGDARYAQKFQGRVTITADESTSTAYMELSSLRSEDTAVYYCARSSYTILTTYYNYNAMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFDYKDDDDKGAPHHHHHH

SEQ ID NO: 35 - protein, 214 aa, Artificial Sequence; identical to SEQ ID NO: 30
  Description of Artificial Sequence Synthetic polypeptide

SEQ ID NO: 36 - protein, 247 aa, Artificial Sequence
  Description of Artificial Sequence Synthetic polypeptide
QVQLVQSGAEVKKPGSSVKVSCKASGGTFSTYAISWVRQAPGQGLEWMGEIIPIFGEADYAQKFQGRVTITADESTSTAYMELSSLRSEDTAVYYCARSSYTILTTYYNYNAMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFDYKDDDDKGAPHHHHHH

SEQ ID NO: 37 - protein, 214 aa, Artificial Sequence; identical to SEQ ID NO: 30
  Description of Artificial Sequence Synthetic polypeptide

SEQ ID NO: 38 - protein, 247 aa, Artificial Sequence
  Description of Artificial Sequence Synthetic polypeptide
QVQLVQSGAEVKKPGSSVKVSCKASGGTFSTYAISWVRQAPGQGLEWMGKIIPIFGDADYAQKFQGRVTITADESTSTAYMELSSLRSEDTAVYYCARSSYTILTTYYNYNAMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFDYKDDDDKGAPHHHHHH

SEQ ID NO: 39 - protein, 214 aa, Artificial Sequence; identical to SEQ ID NO: 30
  Description of Artificial Sequence Synthetic polypeptide

SEQ ID NO: 40 - protein, 247 aa, Artificial Sequence
  Description of Artificial Sequence Synthetic polypeptide
QVQLVQSGAEVKKPGSSVKVSCKASGGTFSTYAISWVRQAPGQGLEWMGQIIPIFGDADYAQKFQGRVTITADESTSTAYMELSSLRSEDTAVYYCARSSYTILTTYYNYNAMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFDYKDDDDKGAPHHHHHH

SEQ ID NO: 41 - protein, 214 aa, Artificial Sequence; identical to SEQ ID NO: 30
  Description of Artificial Sequence Synthetic polypeptide

SEQ ID NO: 42 - protein, 247 aa, Artificial Sequence
  Description of Artificial Sequence Synthetic polypeptide
QVQLVQSGAEVKKPGSSVKVSCKASGGTFSTYAISWVRQAPGQGLEWMGSIIPIFGDADYAQKFQGRVTITADESTSTAYMELSSLRSEDTAVYYCARSSYTILTTYYNYNAMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFDYKDDDDKGAPHHHHHH

SEQ ID NO: 43 - protein, 213 aa, Artificial Sequence; identical to SEQ ID NO: 26
  Description of Artificial Sequence Synthetic polypeptide

SEQ ID NO: 44 - protein, 249 aa, Artificial Sequence
  Description of Artificial Sequence Synthetic polypeptide
QVQLVESGGGLVQPGGSLRLSCAASGFTFNNAWMSWVRQAPGKGLEWVSLIEAKEANGATSYAAGVKGRFTISRDNSKNTLYLQMNSLRAEDTAVYYCARKGMSFYNNKYAKSMFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFDYKDDDDKGAPHHHHHH

SEQ ID NO: 45 - protein, 213 aa, Artificial Sequence; identical to SEQ ID NO: 26
  Description of Artificial Sequence Synthetic polypeptide

SEQ ID NO: 46 - protein, 249 aa, Artificial Sequence
  Description of Artificial Sequence Synthetic polypeptide
QVQLVESGGGLVQPGGSLRLSCAASGFTFNNAWMSWVRQAPGKGLEWVSQIEAKQPGGATSYAASVKGRFTISRDNSKNTLYLQMNSLRAEDTAVYYCARKGMSFYNNKYAKSMFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFDYKDDDDKGAPHHHHHH

SEQ ID NO: 47 - protein, 213 aa, Artificial Sequence; identical to SEQ ID NO: 26
  Description of Artificial Sequence Synthetic polypeptide

SEQ ID NO: 48 - protein, 249 aa, Artificial Sequence
  Description of Artificial Sequence Synthetic polypeptide
QVQLVESGGGLVQPGGSLRLSCAASGFTFNNAWMSWVRQAPGKGLEWVSTIEAKMPNGATFYAAPVKGRFTISRDNSKNTLYLQMNSLRAEDTAVYYCARKGMSFYNNKYAKSMFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFDYKDDDDKGAPHHHHHH

SEQ ID NO: 49 - protein, 213 aa, Artificial Sequence; identical to SEQ ID NO: 26
  Description of Artificial Sequence Synthetic polypeptide

SEQ ID NO: 50 - protein, 247 aa, Artificial Sequence
  Description of Artificial Sequence Synthetic polypeptide
QVQLVESGGGLVQPGGSLRLSCAASGFTFNNAWMSWVRQAPGKGLEWVSAINAKGQFTSYADSVKGRFTISRDNSKNTLYLQMNSLRAEDTAVYYCARKGMSFYNNKYAKSMFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFDYKDDDDKGAPHHHHHH

SEQ ID NO: 51 - protein, 213 aa, Artificial Sequence; identical to SEQ ID NO: 26
  Description of Artificial Sequence Synthetic polypeptide

SEQ ID NO: 52 - protein, 247 aa, Artificial Sequence
  Description of Artificial Sequence Synthetic polypeptide
QVQLVESGGGLVQPGGSLRLSCAASGFTFNNAWMSWVRQAPGKGLEWVSGISADGVHKFYADSVKGRFTISRDNSKNTLYLQMNSLRAEDTAVYYCARKGMSFYNNKYAKSMFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFDYKDDDDKGAPHHHHHH

SEQ ID NO: 53 - protein, 213 aa, Artificial Sequence; identical to SEQ ID NO: 26
  Description of Artificial Sequence Synthetic polypeptide

SEQ ID NO: 54 - protein, 247 aa, Artificial Sequence
  Description of Artificial Sequence Synthetic polypeptide
QVQLVESGGGLVQPGGSLRLSCAASGFTFNNAWMSWVRQAPGKGLEWVSNINVQGDYTGYADSVKGRFTISRDNSKNTLYLQMNSLRAEDTAVYYCARKGMSFYNNKYAKSMFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFDYKDDDDKGAPHHHHHH

SEQ ID NO: 55 - protein, 216 aa, Artificial Sequence
  Description of Artificial Sequence Synthetic polypeptide
DIQMTQSPSSLSASVGDRVTITCRASQNINNFLNWYQQKPGKAPKLLIYGSSILQSGVPSRFSGSGSGTDFTLTISSLQPEDFATYYCSQDHFYPPSGTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 56 - protein, 247 aa, Artificial Sequence
  Description of Artificial Sequence Synthetic polypeptide
QVQLVQSGAEVKKPGSSVKVSCKASGGTFSTYAISWVRQAPGQGLEWMGRIIPIFGTANYAQKFQGRVTITADESTSTAYMELSSLRSEDTAVYYCARSSYTILTTYYNYNAMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFDYKDDDDKGAPHHHHHH

SEQ ID NO: 57 - protein, 215 aa, Artificial Sequence
  Description of Artificial Sequence Synthetic polypeptide
DIQMTQSPSSLSASVGDRVTITCRASQNINNFLNWYQQKPGKAPKLLIYGSSILQSGVPSRFSGSGSGTDFTLTISSLQPEDFATYYCQQNDFYSPSTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 58 - protein, 247 aa, Artificial Sequence; identical to SEQ ID NO: 56
  Description of Artificial Sequence Synthetic polypeptide

SEQ ID NO: 59 - protein, 215 aa, Artificial Sequence
  Description of Artificial Sequence Synthetic polypeptide
DIQMTQSPSSLSASVGDRVTITCRASQNINNFLNWYQQKPGKAPKLLIYGSSILQSGVPSRFSGSGSGTDFTLTISSLQPEDFATYYCQQYDFYLSGTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 60 - protein, 247 aa, Artificial Sequence; identical to SEQ ID NO: 56
  Description of Artificial Sequence Synthetic polypeptide

SEQ ID NO: 61 - protein, 214 aa, Artificial Sequence
  Description of Artificial Sequence Synthetic polypeptide
DIQMTQSPSSLSASVGDRVTITCRASQNINNFLNWYQQKPGKAPKLLIYGSSILQSGVPSRFSGSGSGTDFTLTISSLQPEDFATYYCIQTFFLPDTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 62 - protein, 247 aa, Artificial Sequence; identical to SEQ ID NO: 56
  Description of Artificial Sequence Synthetic polypeptide

SEQ ID NO: 63 - protein, 214 aa, Artificial Sequence
  Description of Artificial Sequence Synthetic polypeptide
DIQMTQSPSSLSASVGDRVTITCRASQNINNFLNWYQQKPGKAPKLLIYGSSILQSGVPSRFSGSGSGTDFTLTISSLQPEDFATYYCQQQDNLPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 64 - protein, 246 aa, Artificial Sequence
  Description of Artificial Sequence Synthetic polypeptide
QVQLVQSGAEVKKPGSSVKVSCKASGGTFSTYAISWVRQAPGQGLEWMGRIIPIFGTANYAQKFQGRVTITADESTSTAYMELSSLRSEDTAVYYCARSSYTILTTYYNYNAMDNWGQGTLVTVSSASTKGPSVFPLAPSSKSTGGTAALGCLVKDYFPEPVTVSWNSGALTSGVHTFPAVLQSSGLYSLSSVVTVPSSSLGTQTYICNVNHKPSNTKVDKKVEPKSEFDYKDDDDKGAPHHHHHH

SEQ ID NO: 65 - protein, 213 aa, Artificial Sequence
  Description of Artificial Sequence Synthetic polypeptide
DIQMTQSPSSLSASVGDRVTITCRASQNINNFLNWYQQKPGKAPKLLIYGSSILQSGVPSRFSGSGSGTDFTLTISSLQPEDFATYYCQQESYVVTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 66 - protein, 247 aa, Artificial Sequence; identical to SEQ ID NO: 56
  Description of Artificial Sequence Synthetic polypeptide

SEQ ID NO: 67 - protein, 213 aa, Artificial Sequence
  Description of Artificial Sequence Synthetic polypeptide
DIQMTQSPSSLSASVGDRVTITCRASQNINNFLNWYQQKPGKAPKLLIYGSSILQSGVPSRFSGSGSGTDFTLTISSLQPEDFATYYCQQTHSQSTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 68 - protein, 247 aa, Artificial Sequence; identical to SEQ ID NO: 56
  Description of Artificial Sequence Synthetic polypeptide

SEQ ID NO: 69 - protein, 213 aa, Artificial Sequence
  Description of Artificial Sequence Synthetic polypeptide
DIELTQPPSVSVAPGQTARISCSGDNLGKKYVHWYQQKPGQAPVLVIYYDSKRPSGIPERFSGSNSGNTATLTISGTQAEDEADYYCQAWGAGSHQMVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEA

SEQ ID NO: 70 - protein, 247 aa, Artificial Sequence
  Description of Artificial Sequence Synthetic polypeptide
QVQLVESGGGLVQPGGSLRLSCAASGFTFNNAWMSWVRQAPGKGLEWVSNITSSSSMTHYADSVKGRFTISRDNSKNTLYLQMNSLRAEDTAVYYCARKGMSFYNNKYAKSMFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFDYKDDDDKGAPHHHHHH

SEQ ID NO: 71 - protein, 213 aa, Artificial Sequence
  Description of Artificial Sequence Synthetic polypeptide
DIELTQPPSVSVAPGQTARISCSGDNLGKKYVHWYQQKPGQAPVLVIYYDSKRPSGIPERFSGSNSGNTATLTISGTQAEDEADYYCQAYDVNYMQDVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEA

SEQ ID NO: 72 - protein, 247 aa, Artificial Sequence
  Description of Artificial Sequence Synthetic polypeptide
QVQLVESGGGLVQPGGSLRLSCAASGFTFSNSWMSWVRQAPGKGLEWVSNITSSSSMTHYADSVKGRFTISRDNSKNTLYLQMNSLRAEDTAVYYCARKGMSFYNNKYAKSMFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFDYKDDDDKGAPHHHHHH

SEQ ID NO: 73 - protein, 213 aa, Artificial Sequence
  Description of Artificial Sequence Synthetic polypeptide
DIELTQPPSVSVAPGQTARISCSGDNLGKKYVHWYQQKPGQAPVLVIYYDSKRPSGIPERFSGSNSGNTATLTISGTQAEDEADYYCQTWTLSHMGNVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEA

SEQ ID NO: 74 - protein, 247 aa, Artificial Sequence; identical to SEQ ID NO: 70
  Description of Artificial Sequence Synthetic polypeptide

SEQ ID NO: 75 - protein, 213 aa, Artificial Sequence
  Description of Artificial Sequence Synthetic polypeptide
DIELTQPPSVSVAPGQTARISCSGDNLGKKYVHWYQQKPGQAPVLVIYYDSKRPSGIPERFSGSNSGNTATLTISGTQAEDEADYYCSSWDSNVVSIVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEA

SEQ ID NO: 76 - protein, 247 aa, Artificial Sequence; identical to SEQ ID NO: 70
  Description of Artificial Sequence Synthetic polypeptide

SEQ ID NO: 77 - protein, 213 aa, Artificial Sequence
  Description of Artificial Sequence Synthetic polypeptide
DIELTQPPSVSVAPGQTARISCSGDNLGKKYVHWYQQKPGQAPVLVIYYDSKRPSGIPERFSGSNSGNTATLTISGTQAEDEADYYCSTYDVGSDYYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEA

SEQ ID NO: 78 - protein, 247 aa, Artificial Sequence; identical to SEQ ID NO: 72
  Description of Artificial Sequence Synthetic polypeptide

SEQ ID NO: 79 - protein, 212 aa, Artificial Sequence
  Description of Artificial Sequence Synthetic polypeptide
DIELTQPPSVSVAPGQTARISCSGDNLGKKYVHWYQQKPGQAPVLVIYYDSKRPSGIPERFSGSNSGNTATLTISGTQAEDEADYYCQSYAGSFFDVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEA

SEQ ID NO: 80 - protein, 247 aa, Artificial Sequence; identical to SEQ ID NO: 70
  Description of Artificial Sequence Synthetic polypeptide

SEQ ID NO: 81 - protein, 213 aa, Artificial Sequence; identical to SEQ ID NO: 73
  Description of Artificial Sequence Synthetic polypeptide

SEQ ID NO: 82 - protein, 247 aa, Artificial Sequence; identical to SEQ ID NO: 50
  Description of Artificial Sequence Synthetic polypeptide

SEQ ID NO: 83 - protein, 212 aa, Artificial Sequence
  Description of Artificial Sequence Synthetic polypeptide
DIELTQPPSVSVAPGQTARISCSGDNLGKKYVHWYQQKPGQAPVLVIYYDSKRPSGIPERFSGSNSGNTATLTISGTQAEDEADYYCQAWGAGSHQMVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVPTEA

SEQ ID NO: 84 - protein, 245 aa, Artificial Sequence
  Description of Artificial Sequence Synthetic polypeptide
QVQLVESGGGLVQPGGSLRLSCAASGFTFNNAWMSWVRQAPGKGLEWVSAINAKGQFTSYADSVKGRFTISRDNSKNTLYLQMNSLRAEDTAVYYCARKGMSFYNNKYAKSMFDLWGQGTLVTVSSASTKGPSVFPLAPSSKSTSGGTAALGCLVKDYFPVTVSWNSGALTSGVHTFPAVLQSSGLYSLSSVVTVPSSSLGTQTYICNVNHKPSNTKVDKKVEPKSEFDYKDDDDKGAPHHHHHH

SEQ ID NO: 85 - protein, 215 aa, Artificial Sequence
  Description of Artificial Sequence Synthetic polypeptide
DIQMTQSPSSLSASVGDRVTITCRASQNINNFLNWYQQKPGKAPKLLIYGSSILQSGVPSRFSGSGSGTDFTLTISSLQPEDFATYYCSQDHFYPPSGTFGQGTKVEIKRTVAAPSVFIFPPSDEQLKGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 86 - protein, 247 aa, Artificial Sequence; identical to SEQ ID NO: 38
  Description of Artificial Sequence Synthetic polypeptide

SEQ ID NO: 87 - protein, 215 aa, Artificial Sequence; identical to SEQ ID NO: 59
  Description of Artificial Sequence Synthetic polypeptide

SEQ ID NO: 88 - protein, 247 aa, Artificial Sequence; identical to SEQ ID NO: 38
  Description of Artificial Sequence Synthetic polypeptide

SEQ ID NO: 89 - protein, 215 aa, Artificial Sequence; identical to SEQ ID NO: 57
  Description of Artificial Sequence Synthetic polypeptide

SEQ ID NO: 90 - protein, 247 aa, Artificial Sequence; identical to SEQ ID NO: 40
  Description of Artificial Sequence Synthetic polypeptide

SEQ ID NO: 91 - protein, 215 aa, Artificial Sequence; identical to SEQ ID NO: 59
  Description of Artificial Sequence Synthetic polypeptide

SEQ ID NO: 92 - protein, 247 aa, Artificial Sequence; identical to SEQ ID NO: 40
  Description of Artificial Sequence Synthetic polypeptide

SEQ ID NO: 93 - protein, 214 aa, Artificial Sequence
  Description of Artificial Sequence Synthetic polypeptide
DIELTQPPSVSVAPGQTARISCSGDNLGKKYVHWYQQKPGQAPVLVIYYDSKRPSGIPERFSGSNSGNTATLTISGTQAEDEADYYCSAWTTTMMHIVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 94 - protein, 455 aa, Artificial Sequence
  Description of Artificial Sequence Synthetic polypeptide
QVQLVESGGGLVQPGGSLRLSCAASGFTFNNAWMSWVRQAPGKGLEWVSQIEAKQPGGATSYAASVKGRFTISRDNSKNTLYLQMNSLRAEDTAVYYCARKGMSFYNNKYAKSMFDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 95 - protein, 214 aa, Artificial Sequence; identical to SEQ ID NO: 93
  Description of Artificial Sequence Synthetic polypeptide

SEQ ID NO: 96 - protein, 455 aa, Artificial Sequence
  Description of Artificial Sequence Synthetic polypeptide
QVQLVESGGGLVQPGGSLRLSCAASGFTFNNAWMSWVRQAPGKGLEWVSTIEAKMPNGATFYAAPVKGRFTISRDNSKNTLYLQMNSLRAEDTAVYYCARKGMSFYNNKYAKSMFDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 97 - protein, 214 aa, Artificial Sequence; identical to SEQ ID NO: 93
  Description of Artificial Sequence Synthetic polypeptide

SEQ ID NO: 98 - protein, 453 aa, Artificial Sequence
  Description of Artificial Sequence Synthetic polypeptide
QVQLVESGGGLVQPGGSLRLSCAASGFTFNNAWMSWVRQAPGKGLEWVSGISADGVHKFYADSVKGRFTISRDNSKNTLYLQMNSLRAEDTAVYYCARKGMSFYNNKYAKSMFDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 99 - protein, 214 aa, Artificial Sequence; identical to SEQ ID NO: 93
  Description of Artificial Sequence Synthetic polypeptide

SEQ ID NO: 100 - protein, 453 aa, Artificial Sequence
  Description of Artificial Sequence Synthetic polypeptide
QVQLVESGGGLVQPGGSLRLSCAASGFTFNNAWMSWVRQAPGKGLEWVSNINVQGDYTGYADSVKGRFTISRDNSKNTLYLQMNSLRAEDTAVYYCARKGMSFYNNKYAKSMFDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 101 - protein, 215 aa, Artificial Sequence
  Description of Artificial Sequence Synthetic polypeptide
DIQMTQSPSSLSASVGDRVTITCRASQNINNFLNWYQQKPGKAPKLLIYGSSILQSGVPSRFSGSGSGTDFTLTISSLQPEDFATYYCQQNDFYSPSTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 453 aa, Artificial Sequence
  Description of Artificial Sequence Synthetic polypeptide
QVELVQSGAEVKKPGSSVKVSCKASGGTFSTYAISWVRQAPGQGLEWMGRIIPIFGTANYAQKFQGRVTITADESTSTAYMELSSLRSEDTAVYYCARSSYTILTTYYNYNAMDNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 103 - protein, 215 aa, Artificial Sequence
  Description of Artificial Sequence Synthetic polypeptide
DIQMTQSPSSLSASVGDRVTITCRASQNINNFLNWYQQKPGKAPKLLIYGSSILQSGVPSRFSGSGSGTDFTLTISSLQPEDFATYYCQQYDFYLSGTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 453 aa, Artificial Sequence; identical to SEQ ID NO: 102
  Description of Artificial Sequence Synthetic polypeptide

SEQ ID NO: 105 - protein, 214 aa, Artificial Sequence
  Description of Artificial Sequence Synthetic polypeptide
DIELTQPPSVSVAPGQTARISCSGDNLGKKYVHWYQQKPGQAPVLVIYYDSKRPSGIPERFSGSNSGNTATLTISGTQAEDEADYYCQTWTLSHMGNVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 106 - protein, 453 aa, Artificial Sequence
  Description of Artificial Sequence Synthetic polypeptide
QVELVESGGGLVQPGGSLRLSCAASGFTFNNAWMSWVRQAPGKGLEWVSAINAKGQFTSYADSVKGRFTISRDNSKNTLYLQMNSLRAEDTAVYYCARKGMSFYNNKYAKSMFDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 107 - protein, 214 aa, Artificial Sequence
  Description of Artificial Sequence Synthetic polypeptide
DIELTQPPSVSVAPGQTARISCSGDNLGKKYVHWYQQKPGQAPVLVIYYDSKRPSGIPERFSGSNSGNTATLTISGTQAEDEADYYCQAWGAGSHQMVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 108 - protein, 453 aa, Artificial Sequence; identical to SEQ ID NO: 106
  Description of Artificial Sequence Synthetic polypeptide

SEQ ID NO: 109 - protein, 216 aa, Artificial Sequence
  Description of Artificial Sequence Synthetic polypeptide
DIQMTQSPSSLSASVGDRVTITCRASQNINNFLNWYQQKPGKAPKLLIYGSSILQSGVPSRFSGSGSGTDFTLTISSLQPEDFATYYCSQDHFYPPSGTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 453 aa, Artificial Sequence
  Description of Artificial Sequence Synthetic polypeptide
QVELVQSGAEVKKPGSSVKVSCKASGGTFSTYAISWVRQAPGQGLEWMGKIIPIFGDADYAQKFQGRVTITADESTSTAYMELSSLRSEDTAVYYCARSSYTILTTYYNYNAMDNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 111 - protein, 215 aa, Artificial Sequence; identical to SEQ ID NO: 103
  Description of Artificial Sequence Synthetic polypeptide

SEQ ID NO: 112 - protein, 453 aa, Artificial Sequence; identical to SEQ ID NO: 110
  Description of Artificial Sequence Synthetic polypeptide

SEQ ID NO: 113 - protein, 215 aa, Artificial Sequence; identical to SEQ ID NO: 101
  Description of Artificial Sequence Synthetic polypeptide

SEQ ID NO: 114 - protein, 453 aa, Artificial Sequence
  Description of Artificial Sequence Synthetic polypeptide
QVELVQSGAEVKKPGSSVKVSCKASGGTFSTYAISWVRQAPGQGLEWMGQIIPIFGDADYAQKFQGRVTITADESTSTAYMELSSLRSEDTAVYYCARSSYTILTTYYNYNAMDNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 115 - protein, 215 aa, Artificial Sequence; identical to SEQ ID NO: 103
  Description of Artificial Sequence Synthetic polypeptide

SEQ ID NO: 116 - protein, 453 aa, Artificial Sequence; identical to SEQ ID NO: 114
  Description of Artificial Sequence Synthetic polypeptide

SEQ ID NO: 117 - protein, 214 aa, Artificial Sequence; identical to SEQ ID NO: 107
  Description of Artificial Sequence Synthetic polypeptide

SEQ ID NO: 118 - protein, 456 aa, Artificial Sequence
  Description of Artificial Sequence Synthetic polypeptide
QVELVESGGGLVQPGGSLRLSCAASGFTFNNAWMSWVRQAPGKGLEWVSAINAKGQFTSYADSVKGRFTISRDNSKNTLYLQMNSLRAEDTAVYYCARKGMSFYNNKYAKSMFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 10 aa, Artificial Sequence
  Description of Artificial Sequence Synthetic peptide
SQDHFYPPSG

SEQ ID NO: 120 - protein, 9 aa, Artificial Sequence
  Description of Artificial Sequence Synthetic peptide
QQNDFYSPS

SEQ ID NO: 121 - protein, 9 aa, Artificial Sequence
  Description of Artificial Sequence Synthetic peptide
QQYDFYLSG

SEQ ID NO: 122 - protein, 8 aa, Artificial Sequence
  Description of Artificial Sequence Synthetic peptide
IQTFFLPD

SEQ ID NO: 123 - protein, 8 aa, Artificial Sequence
  Description of Artificial Sequence Synthetic peptide
QQQDNLPR

SEQ ID NO: 124 - protein, 8 aa, Artificial Sequence
  Description of Artificial Sequence Synthetic peptide
QQESYVVT

SEQ ID NO: 125 - protein, 8 aa, Artificial Sequence
  Description of Artificial Sequence Synthetic peptide
QQTHSQST

SEQ ID NO: 126 - protein, 10 aa, Artificial Sequence
  Description of Artificial Sequence Synthetic peptide
QAWGAGSHQM

SEQ ID NO: 127 - protein, 10 aa, Artificial Sequence
  Description of Artificial Sequence Synthetic peptide
QAYDVNYMQD

SEQ ID NO: 128 - protein, 10 aa, Artificial Sequence
  Description of Artificial Sequence Synthetic peptide
QTWTLSHMGN

SEQ ID NO: 129 - protein, 10 aa, Artificial Sequence
  Description of Artificial Sequence Synthetic peptide
SSWDSNVVSI

SEQ ID NO: 130 - protein, 10 aa, Artificial Sequence
  Description of Artificial Sequence Synthetic peptide
STYDVGSDYY

SEQ ID NO: 131 - protein, 9 aa, Artificial Sequence
  Description of Artificial Sequence Synthetic peptide
QSYAGSFFD

SEQ ID NO: 132 - protein, 10 aa, Artificial Sequence
  Description of Artificial Sequence Synthetic peptide
GFTFNNAWMS

SEQ ID NO: 133 - protein, 10 aa, Artificial Sequence
  Description of Artificial Sequence Synthetic peptide
GFTFSNSWMS

SEQ ID NO: 134 - protein, 20 aa, Artificial Sequence
  Description of Artificial Sequence Synthetic peptide
WMGAIIPIWGDAWYAQKFQG

SEQ ID NO: 135 - protein, 20 aa, Artificial Sequence
  Description of Artificial Sequence Synthetic peptide
WMGDIIPIWGDARYAQKFQG

SEQ ID NO: 136 - protein, 20 aa, Artificial Sequence
  Description of Artificial Sequence Synthetic peptide
WMGEIIPIFGEADYAQKFQG

SEQ ID NO: 137 - protein, 20 aa, Artificial Sequence
  Description of Artificial Sequence Synthetic peptide
WMGKIIPIFGDADYAQKFQG

SEQ ID NO: 138 - protein, 20 aa, Artificial Sequence
  Description of Artificial Sequence Synthetic peptide
WMGQIIPIFGDADYAQKFQG

SEQ ID NO: 139 - protein, 20 aa, Artificial Sequence
  Description of Artificial Sequence Synthetic peptide
WMGSIIPIFGDADYAQKFQG

SEQ ID NO: 140 - protein, 22 aa, Artificial Sequence
  Description of Artificial Sequence Synthetic peptide
WVSLIEAKEANGATSYAAGVKG

SEQ ID NO: 141 - protein, 22 aa, Artificial Sequence
  Description of Artificial Sequence Synthetic peptide
WVSQIEAKQPGGATSYAASVKG

SEQ ID NO: 142 - protein, 22 aa, Artificial Sequence
  Description of Artificial Sequence Synthetic peptide
WVSTIEAKMPNGATFYAAPVKG

SEQ ID NO: 143 - protein, 20 aa, Artificial Sequence
  Description of Artificial Sequence Synthetic peptide
WVSAINAKGQFTSYADSVKG

SEQ ID NO: 144 - protein, 20 aa, Artificial Sequence
  Description of Artificial Sequence Synthetic peptide
WVSGISADGVHKFYADSVKG

SEQ ID NO: 145 - protein, 20 aa, Artificial Sequence
  Description of Artificial Sequence Synthetic peptide
WVSNINVQGDYTGYADSVKG

SEQ ID NO: 146 - protein, 17 aa, Artificial Sequence
  Description of Artificial Sequence Synthetic peptide
AINAKGQFTSYADSVKG

SEQ ID NO: 147 - protein, 17 aa, Artificial Sequence
  Description of Artificial Sequence Synthetic peptide
KIIPIFGDADYAQKFQG

SEQ ID NO: 148 - protein, 17 aa, Artificial Sequence
  Description of Artificial Sequence Synthetic peptide
QIIPIFGDADYAQKFQG

SEQ ID NO: 149 - protein, 10 aa, Artificial Sequence
  Description of Artificial Sequence Synthetic peptide
QAWGAGSHQM

SEQ ID NO: 150 - protein, 250 aa, Artificial Sequence
  Description of Artificial Sequence Synthetic polypeptide
QVQLVESGGGLVQPGGSLRLSCAASGFTFNNSWMSWVRQAPGKGLEWVSNITSSSSMTHYADSVKGRFTISRDNSKNTLYLQMNSLRAEDTAVYYCARKGMSFYNNKYAKSMFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFEQKLISEEDLNGAPHHHHHH

SEQ ID NO: 151 - protein, 6 aa, Artificial Sequence
  Description of Artificial Sequence Synthetic 6xHis tag
HHHHHH